(19) United States
(12) Patent Application Publication
(10) Pub. No.: US 2024/0423558 A1
(43) Pub. Date: Dec. 26, 2024

(54) PREDICTIVE LONG HYPERGLYCEMIA ALERTS

(71) Applicant: ABBOTT DIABETES CARE INC., Alameda, CA (US)

(72) Inventors: Erwin S. Budiman, Fremont, CA (US); Sediqeh Samadi, Emeryville, CA (US); Aparajita Bhattacharya, Dublin, CA (US); Gary A. Hayter, Oakland, CA (US)

(21) Appl. No.: 18/752,220

(22) Filed: Jun. 24, 2024

Related U.S. Application Data
(60) Provisional application No. 63/545,433, filed on Oct. 24, 2023.
(60) Provisional application No. 63/523,238, filed on Jun. 26, 2023.

(51) Int. Cl. A61B 5/00; A61B 5/145

(52) U.S. Cl. CPC A61B 5/746; A61B 5/14503; A61B 5/14532; A61B 5/7264; A61B 5/7275; A61B 5/7435; A61B 2560/045

(57) ABSTRACT

Improved alarms for analyte monitoring systems are provided. For example, disclosed herein are various embodiments of methods, systems, and interfaces for long hyperglycemic alerts or pre-alerts. In addition, various embodiments of methods, systems, and interfaces for modified high glucose alerts, which predict if the high glucose episode is associated with a recently administered medication dose are described. Also, systems that include both high glucose alerts and long hyperglycemic alert options are described, in addition to enabling and disabling or suppressing certain alerts under various conditions.

### CROSS-REFERENCE TO RELATED APPLICATIONS

This application claims priority to U.S. Provisional Application No. 63/545,433, filed Oct. 24, 2023, and U.S. Provisional Application No. 63/523,238, filed Jun. 26, 2023, both of which are herein expressly incorporated by reference in their entireties for all purposes.

### FIELD

The subject matter described herein relates generally to digital interfaces, user interfaces, and alarms for analyte monitoring systems, as well as systems, methods, and devices relating thereto.

### BACKGROUND

The detection and/or monitoring of analyte levels, such as glucose, ketones, lactate, oxygen, hemoglobin A1C, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with the bodily fluid. The analyte monitoring system may also be configured to transmit analyte data and/or alarms to another device, from which a caregiver such as, for example, a parent, a spouse, or a health care provider (“HCP”), can review the data and make therapy decisions. Furthermore, the benefits of analyte monitoring systems are not limited to persons with diabetes. For instance, analyte monitoring systems can provide useful information and insights to individuals interested in improving their health and wellness. As one example, to improve their sports performance, athletes can utilize a sensor control device worn on the body to collect data relating to one or more analytes such as, for example, glucose and/or lactate. Other non-medical applications for analyte monitoring systems are possible and described in further detail below.

In many existing glucose monitoring applications, when glucose value crosses a high threshold, high glucose alarm is enunciated. Diabetes patients, however, often experience a significant post-prandial spike in their glucose levels, even when using the appropriate medications (e.g., rapid-acting insulin) to lower post-prandial glucose levels. For some patients, even when a proper amount of insulin is taken to cover their meal, they will often experience a relatively short glucose spike. This short spike, which is caused by a mismatch between the glucose response dynamics due to the meal compared to that due to the insulin, is generally accepted because the post-prandial glucose is mostly controlled. When the post-prandial glucose spike is short, the proper action for the patient to take is to wait to make sure the glucose levels fall. The patient should not inject more insulin. Standard of care guidance, as provided by the Endocrine society, recommends that the patients wait for some time (e.g., two or more hours) after the meal-time insulin injection; if their glucose levels are still high and not falling after this time period, then a correction dose may be administered to address their high glucose levels. Injecting insulin any earlier than this is referred to as “stacking” and is associated with a high risk of hypoglycemia. Thus, a high glucose alarm that is outputted right after the patient starts eating not only does not provide any actionable benefit, but it may annoy the patient, and in the worst case, may tempt the patient to inject a correction dose too early.

The current high glucose threshold alarm does not distinguish between a persistently high glucose levels and temporarily high glucose levels. Persistently high glucose levels may be due to insufficient meal insulin dose, a missed long-acting insulin dose, or consuming a meal with a higher glycemic load than initially thought. In contrast, temporarily high glucose levels may be a result of consuming simple carbohydrates with high glycemic index. As a result, when setting their alarm conditions in their glucose monitoring system, the user must deal with frequent, irrelevant alarms from each initial post-meal spike when setting the threshold to a reasonable “stable” post meal threshold target. Being able to promptly and accurately distinguish these two categories can help with reducing postmeal glucose rise.

Another possible mitigation is that the user may be forced to select a very high but ineffective high glucose threshold. Setting a very high glucose alarm threshold, however, may have negative impacts. First, a certain exposure to hyperglycemia has occurred (due to the higher threshold), and second, the larger mismatch between the glucose appearance (due to the meal) and glucose clearance (due to a late dose insulin) increases the risk of post meal hypoglycemia.

Another possible mitigation is the for system to delay the high alert. For example, the alert may not be asserted until the glucose level has exceeded the alert threshold for some period of time, such as 1 hour. While this may prevent the high alert from asserting due to these temporary post-meal spikes in many case, the downside of such a setting is that the sensor system may not be aware of when the last insulin dose was administered, so the high glucose reading may not be due to a recent meal-bolus event, but rather a missed meal dose or some other reason where a correction insulin dose is warranted. In these cases, the patient may be at risk of diabetic ketoacidosis (DKA) where timely administration of insulin is necessary. In any case, the patient will be experiencing poor glucose control, along with its long-term deleterious effects.

Thus, a need exists for methods for estimating and notifying a user of long hyperglycemic events.

### SUMMARY

Provided herein are example embodiments of digital and user interfaces for analyte monitoring systems. Aspects of the inventions are set out in the independent claims and preferred features are set out in the dependent claims. Preferred features of each aspect may be provided in combination with each other within particular embodiments and may also be provided in combination with other aspects. According to some embodiments, methods, systems, and interfaces relating to determining long, high glucose alerts or pre-alerts are described. In other embodiments, methods, systems, and interfaces for determining high glucose alarms, including determining if a glucose pattern of a potential hyperglycemic event is associated with an administered medication dose or not are described. In still other embodiments, methods, systems, and interfaces relating to options for enabling and disabling high glucose alarms and long, high glucose alerts or pre-alerts under different conditions are described.

A long hyperglycemic event referred to herein is high glucose levels for a long duration. A long duration may be a duration of at least 1 hour, alternatively at least 1.5 hours, alternatively at least 2 hours, alternatively at least 2.5 hours, alternatively at least 3 hours, alternatively at least 3.5 hours, alternatively at least 4 hours. High glucose levels may be glucose levels of at least 140 mg/L, alternatively at least 160 mg/L, alternatively at least 180 mg/L, alternatively at least 200 mg/L, alternatively at least 220 mg/dL, alternatively at least 240 mg/dL, alternatively at least 260 mg/dL. In one embodiment, a long hyperglycemic event referred to herein may be glucose levels of at least 180 mg/L for a duration of at least 2 hours. The present invention involves identifying/determining a potential/possible long hyperglycemic event occurring in the future. In other words, the present invention involves predicting the occurrence of a long hyperglycemic event in the future. The potential/possible long hyperglycemic event may be predicted using the received glucose data indicative of one or more glucose levels.

In many embodiments, an analyte monitoring system includes: a sensor control device comprising an analyte sensor, wherein at least a portion of the analyte sensor is configured to be in fluid contact with a bodily fluid of a subject; and a reader device, comprising: wireless communication circuitry configured to receive data indicative of one or more glucose levels; and one or more processors coupled to a memory, the memory storing instructions that, when executed by the one or more processors, cause the one or more processors to: predict a hyperglycemic event based on the data indicative of one or more glucose levels; predict a total duration for the predicted hyperglycemic event and compare the total duration to a threshold to determine if the predicted hyperglycemic event is a long hyperglycemic event; and if the predicted hyperglycemic event is a long hyperglycemic event, then output an alert regarding the long hyperglycemic event.

In some embodiments, the hyperglycemic is predicted when a glucose level is above the threshold. In some embodiments, the threshold value may be at least 140 mg/L, alternatively at least 160 mg/L, alternatively at least 180 mg/L, alternatively at least 200 mg/L, alternatively at least 220 mg/dL, alternatively at least 240 mg/dL, alternatively at least 260 mg/dL.

In some embodiments, the hyperglycemic event is predicted when a start of a rise in glucose levels is detected.

In some embodiments, the instructions, when executed by the one or more processors, cause the one or more processors to estimate a plurality of future glucose levels.

In some embodiments, the total duration for the predicted hyperglycemic event is determined based on an amount of time future glucose levels of the plurality of future glucose levels are above the threshold.

In some embodiments, the estimated plurality of future glucose levels comprises a plurality of estimated lower glucose level bounds and a plurality of upper glucose level bounds. The plurality of glucose levels have a spread between a range formed between a lower glucose level bound and an upper glucose level bound. In some embodiments, the total duration for the predicted hyperglycemic event comprises a lower bound total duration and an upper bound total duration.

In some embodiments, the total duration comprises an estimated lower bound total duration and an estimated upper bound total duration. In some embodiments, the lower bound total duration and the upper bound total duration are determined without determining a plurality of estimated lower glucose level bounds and a plurality of upper glucose level bounds.

In some embodiments, the total duration for the predicted hyperglycemic event is estimated based on a time series of a plurality of glucose levels after the determined start of the rise.

In some embodiments, the total duration for the predicted hyperglycemic event is estimated based on a plurality of glucose metrics determined from a period of time. In some embodiments, the glucose metrics may be used to determine how to count the total duration when the glucose time series values (from the estimated future glucose values) goes below and above the threshold several times in recent succession. In other embodiments, the estimated future glucose values may be calculated with upper and lower bounds, and some traces go above and below the threshold several times. A plurality of glucose metrics could help with this determination. For example, one metric may be when glucose values within the total duration of the predicted hyperglycemic event are predicted to have a Time Above Range exceeding 250 mg/dL (TAR250) for at least 30 minutes. In other embodiments, a metric may be whether or not recent similar excursions lasted longer than a predetermined period of time. If recent similar excursions did not, then the metric may reduce the estimated total duration by a scaling factor greater than 0 but less than 1, or reduce the total duration by a pre-determined equation as a function of that metric. The metrics may be determined from glucose levels received and/or predicted. For example, given the timing of the prediction at or slightly before crossing the high glucose threshold, then only glucose levels predicted are involved because none of the recent received glucose levels exceed that threshold. In another example, a metric reflecting glucose rate of change in the past 2 hours may rely solely on the received glucose levels. In another example, a similar metric reflecting a similar 2-hour glucose rate of change until the first crossing of the threshold can include both received glucose and predicted glucose values.

In some embodiments, the plurality of glucose metrics comprises at least one of an average glucose value, a minimum glucose value, a maximum glucose value, and a standard deviation. In some embodiments, the period of time is selected from the group consisting of about 12 hours, about 24 hours, about 1 week, and about 2 weeks.

In some embodiments, the total duration is further estimated based on inputs selected from the group consisting of insulin doses and meals.

In some embodiments, the alert is outputted if the total duration is above a threshold value. In some embodiments, the threshold value may be at least 1 hour, alternatively at least 1.5 hours, alternatively at least 2 hours, alternatively at least 2.5 hours, alternatively at least 3 hours, alternatively at least 3.5 hours, alternatively at least 4 hours.

In some embodiments, the instructions, when executed by the one or more processors, cause the one or more processors to: determine a risk of hypoglycemia during the long hyperglycemic event; and if a risk of hypoglycemia is determined to exist, then suppress the alert regarding the long hyperglycemic event. In some embodiments, determining the risk of hypoglycemia during the long hyperglycemic event comprises determining a glucose pattern. In some embodiments, the glucose pattern comprises a plurality of categories. In some embodiments, the plurality of categories comprises a long high glucose pattern, a glucose pattern with a low glucose risk, and any other pattern, and wherein a risk of hypoglycemia is determined if the glucose pattern is determined to be the glucose pattern with a low glucose risk. The glucose pattern with low glucose risk may correspond to situations where any additional intervention in response to a high glucose alert may increase or cause hypoglycemia, where their glucose levels to drop below a low glucose threshold. The low glucose threshold may be about 80 mg/dL, alternatively about 75 mg/dL, alternatively about 70 mg/dL, alternatively about 65 mg/dL.

In some embodiments, the instructions, when executed by the one or more processors, cause the one or more processors to: determine a probability of the glucose pattern comprising a long high glucose pattern; and determine a time to output the alert based on a comparison of the probability of the glucose pattern comprising the long high glucose pattern to a threshold probability. In some embodiments, the determination of the probability of the glucose pattern comprising a long high glucose pattern is determined based on calculating an area under the curve (AUC) above the high glucose threshold. For example, if the high glucose threshold is set at 180 mg/dL, then the AUC generated from glucose levels above 180 mg/dL may be compared against a pre-determined AUC threshold, or a function that generates the probability using the generated AUC value as the input. An AUC threshold example may be 80 mg/dL-hour, which is equivalent to staying 40 mg/dL above the threshold for 2 hours, or staying 20 mg/dL above the threshold for 4 hours, or other more complicated patterns. In another embodiment, instead of using the same threshold, a second higher threshold, such as 250 mg/dL, may be used to calculate the AUC even when the high glucose threshold is set at a different value, e.g., 180 mg/dL. In this example, while having at least one value crossing the 180 mg/dL is needed to determine the probability of long high glucose pattern, the probability may be low if the AUC above 250 mg/dL is zero or below a certain pre-determined AUC threshold. In another embodiment, while having at least one value crossing the 180 mg/dL is needed to determine the probability of long high glucose pattern, the probability may be low if the time spent above a second threshold, e.g., 240 mg/dL, is below a certain pre-determined time threshold.

In some embodiments, the instructions, when executed by the one or more processors, cause the one or more processors to determine a time to output the alert based at least on the determined total duration. In some embodiments, the hyperglycemic event is predicted when a start of a rise in glucose levels is detected, and wherein the alert is outputted after a time period after the start of the rise in glucose levels. In some embodiments, the time period is based on a population dataset. In some embodiments, the time period is based on a dataset of the subject. In some embodiments, the time period is based on a value of a glucose level after the start of the rise in glucose levels is above a threshold value.

In some embodiments, the determined time to output the alert is when a glucose level is equal to or is above the high glucose threshold.

In some embodiments, the instructions, when executed by the one or more processors, cause the one or more processors to determine an expiration time for the alert, wherein the alert will not be outputted after the expiration time.

In some embodiments, the instructions, when executed by the one or more processors, cause the one or more processors to activate an alarm when a determined glucose level of the one or more glucose levels is above a high glucose alarm threshold.

In some embodiments, the instructions, when executed by the one or more processors, cause the one or more processors to: determine if the subject has acknowledged the outputted alert; and output an additional alert notification after a waiting time if the subject has not acknowledged the outputted alert. In some embodiments, the waiting time is based on the determined total duration.

In some embodiments, the plurality of future glucose levels are estimated based on determined glucose levels from a predetermined time period before the start of the rise in glucose levels.

In some embodiments, the plurality of future glucose levels are estimated based on determined glucose levels from a predetermined time period before a latest determined glucose level based on the received sensor readings. In some embodiments, the predetermined time period is about 4 hours.

In some embodiments, determining the risk of hypoglycemia during the potential long hyperglycemic event comprises determining a glucose pattern, wherein the instructions, when executed by the one or more processors, cause the one or more processors to: determine an area under the curve (AUC) of the plurality of future glucose levels, wherein the glucose pattern is determined to be one of a plurality of categories based on a comparison of the AUC to a threshold AUC. In some embodiments, the plurality of categories comprises a low category, a medium category, and a high category. In some embodiments, the high glucose pattern is categorized based on an area under the curve of the estimated plurality of future glucose levels.

In some embodiments, the instructions, when executed by the one or more processors, cause the one or more processors to: determine it the subject has acknowledged the outputted alert; and output an additional alert notification after a waiting time if the subject has not acknowledged the outputted alert. In some embodiments, the waiting time is based on the determined total duration for the potential long hyperglycemic event above the high threshold.

In some embodiments, the potential long hyperglycemic is detected when a start of a rise in glucose levels is detected, and wherein the instructions, when executed by the one or more processors, cause the one or more processors to: determine an area under the curve of the plurality of future glucose levels after the start of the rise, and wherein the waiting time is based on the determined area under the curve.

In some embodiments, the waiting time may be varied based on an amount of the determined area under the curve.

In many embodiments, a method for monitoring a glucose concentration of a user includes the steps of: receiving measurement data related to the glucose concentration of the user; predicting a hyperglycemic event based on the data indicative of one or more glucose levels; predicting a total duration for the predicted hyperglycemic event and compare the total duration to a threshold to determine if the predicted hyperglycemic event is a long hyperglycemic event; and if the predicted hyperglycemic event is a long hyperglycemic event, then outputting an alert regarding the long hyperglycemic event.

In some embodiments, the hyperglycemic is predicted when a glucose level is above the threshold. In some embodiments, the hyperglycemic event is predicted when a start of a rise in glucose levels is detected.

In some embodiments, the method further includes the step of estimating a plurality of future glucose levels. In some embodiments, the total duration for the predicted hyperglycemic event is determined based on an amount of time future glucose levels of the plurality of future glucose levels are above the threshold. In some embodiments, the estimated plurality of future glucose levels comprises a plurality of estimated lower glucose level bounds and a plurality of upper glucose level bounds. In some embodiments, the total duration for the predicted hyperglycemic event comprises a lower bound total duration and an upper bound total duration.

In some embodiments, the total duration comprises a lower bound total duration and an upper bound total duration. In some embodiments, the lower bound total duration and the upper bound total duration are determined without determining a plurality of estimated lower glucose level bounds and a plurality of estimated upper glucose level bounds.

In some embodiments, the total duration for the predicted hyperglycemic event is estimated based on a time series of a plurality of glucose levels after the determined start of the rise.

In some embodiments, the total duration for the predicted hyperglycemic event is estimated based on a plurality of glucose metrics determined from a period of time. In some embodiments, the plurality of glucose metrics comprises at least one of an average glucose value, a minimum glucose value, a maximum glucose value, and a standard deviation. In some embodiments, the period of time is selected from the group consisting of about 12 hours, about 24 hours, about 1 week, and about 2 weeks.

In some embodiments, the total duration is further estimated based on inputs selected from the group consisting of insulin doses and meals.

In some embodiments, the method further includes the steps of determining a risk of hypoglycemia during the long hyperglycemic event; and if a risk of hypoglycemia is determined to exist, then suppressing the alert regarding the long hyperglycemic event. In some embodiments, determining the risk of hypoglycemia during the long hyperglycemic event comprises determining a glucose pattern. In some embodiments, the glucose pattern comprises a plurality of categories. In some embodiments, the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.

In some embodiments, the method further includes the steps of: determining a probability of the glucose pattern; and determining a time to output the alert based on a comparison of the probability of the glucose pattern to a threshold probability.

In some embodiments, the method further includes the step of determining a time to output the alert based at least on the determined total duration. In some embodiments, the hyperglycemic event is predicted when a start of a rise in glucose levels is detected, and wherein the alert is outputted after a time period after the start of the rise in glucose levels of the predicted hyperglycemic event. In some embodiments, the time period is based on a population dataset. In some embodiments, the time period is based on a dataset of the subject. In some embodiments, the time period is based on a value of a glucose level after the start of the rise in glucose levels is above a threshold value.

In some embodiments, the determined time to output the alert is when a glucose level is equal to or is above the high glucose threshold.

In some embodiments, the method further includes the step of determining an expiration time for the alert, wherein the alert will not be outputted after the expiration time.

In some embodiments, the method further includes the step of activating an alarm when a determined glucose level of the plurality of glucose levels is above a high glucose alarm threshold.

In some embodiments, the method further includes the steps of: determining if the subject has acknowledged the outputted alert; and outputting an additional alert notification after a waiting time if the subject has not acknowledged the outputted alert. In some embodiments, the waiting time is based on the determined total duration.

In some embodiments, the plurality of future glucose levels are estimated based on determined glucose levels from a predetermined time period before the start of the rise in glucose levels.

In some embodiments, the plurality of future glucose levels are estimated based on determined glucose levels from a predetermined time period before a latest determined glucose level based on the received sensor readings. In some embodiments, the predetermined time period is about 4 hours.

In some embodiments, determining the risk of hypoglycemia during the potential long hyperglycemic event comprises determining a glucose pattern, wherein the instructions, when executed by the one or more processors, cause the one or more processors to: determining an area under the curve (AUC) of the plurality of future glucose levels, wherein the glucose pattern is determined to be one of a plurality of categories based on a comparison of the AUC to a threshold AUC.

In some embodiments, the plurality of categories comprises a low category, a medium category, and a high category.

In some embodiments, the high glucose pattern is categorized based on an area under the curve of the estimated plurality of future glucose levels.

In some embodiments, the method further includes the steps of: determining if the subject has acknowledged the outputted alert; and outputting an additional alert notification after a waiting time if the subject has not acknowledged the outputted alert. In some embodiments, the waiting time is based on the determined total duration for the potential long hyperglycemic event above the high threshold.

In some embodiments, the potential long hyperglycemic is detected when a start of a rise in glucose levels is detected, and the method further comprises the step of determining an area under the curve of the plurality of future glucose levels after the start of the rise, and wherein the waiting time is based on the determined area under the curve.

In some embodiments, the waiting time may be varied based on an amount of the determined area under the curve.

In many embodiments, an analyte monitoring system includes a sensor control device comprising an analyte sensor, wherein at least a portion of the analyte sensor is configured to be in fluid contact with a bodily fluid of a subject; and a reader device, comprising: wireless communication circuitry configured to receive data indicative of one ore more glucose levels; and one or more processors coupled to a memory, the memory storing instructions that, when executed by the one or more processors, cause the one or more processors to: predict a hyperglycemic event based on the data indicative of one or more glucose levels; estimate a plurality of future glucose levels; predict a total duration for the hyperglycemic event and compare the total duration to a threshold to determine if the predicted hyperglycemic event is a long hyperglycemic event; and if the predicted hyperglycemic event is a long hyperglycemic event, then output an alert regarding the long hyperglycemic event.

In some embodiments, the hyperglycemic event is predicted when a glucose level is above the threshold.

In some embodiments, the hyperglycemic event is predicted when a start of a rise in glucose levels is detected.

In some embodiments, the plurality of future glucose levels are estimated based on determined glucose levels from a predetermined time period before the start of the rise in glucose levels.

In some embodiments, the plurality of future glucose levels are estimated based on determined glucose levels from a predetermined time period before a latest determined glucose level based on the received sensor readings. In some embodiments, the predetermined time period is about 4 hours.

In some embodiments, the total duration for the hyperglycemic event is predicted based on an amount of time the plurality of future glucose levels are above the threshold.

In some embodiments, the plurality of future glucose levels comprises a plurality of estimated lower glucose level bounds and a plurality of estimated upper glucose level bounds. In some embodiments, the total duration comprises a lower bound total duration and an upper bound total duration.

In some embodiments, the instructions, when executed by the one or more processors, cause the one or more processors to: determine a risk of hypoglycemia during the long hyperglycemic event; and if a risk of hypoglycemia is determined to exist, then suppress the alert regarding the long hyperglycemic event.

In some embodiments, determining the risk of hypoglycemia during the potential long hyperglycemic event comprises determining a glucose pattern, wherein the instructions, when executed by the one or more processors, cause the one or more processors to: determine an area under the curve (AUC) of the plurality of future glucose levels, wherein the glucose pattern is categorized according to one of a plurality of categories based on a comparison of the AUC to a threshold AUC. In some embodiments, the plurality of categories comprises a low category, a medium category, and a high category. In some embodiments, the glucose pattern is categorized as a high glucose pattern based on an area under the curve of the estimated plurality of future glucose levels.

In some embodiments, the instructions, when executed by the one or more processors, cause the one or more processors to: determine it the subject has acknowledged the outputted alert; and output an additional alert notification after a waiting time if the subject has not acknowledged the outputted alert. In some embodiments, the waiting time is based on the determined total duration for the potential long hyperglycemic event above a high threshold. In some embodiments, the hyperglycemic alert is predicted when a start of a rise in glucose levels is detected, and wherein the instructions, when executed by the one or more processors, cause the one or more processors to: determine an area under the curve of the plurality of future glucose levels after the start of the rise, and wherein the waiting time is based on the determined area under the curve.

In some embodiments, the instructions, when executed by the one or more processors, cause the one or more processors to: determine a time to output the alert based at least on the determined total duration. In some embodiments, the hyperglycemic event is detected when a start of a rise in glucose levels is detected, and wherein the determined time to output the alert is after a time period after the start of the rise in glucose levels of the potential long hyperglycemic event. In some embodiments, the time period is based on a population dataset. In some embodiments, the time period is based on a dataset of the subject. In some embodiments, the time period is based on a value of a glucose level after the start of the rise in glucose levels is above a threshold value. the determined time to output the alert is when a glucose level is equal to or is above the high glucose threshold.

In many embodiments, a method for monitoring a glucose concentration of a user includes the steps of: receiving measurement data related to the glucose concentration of the user; predicting a hyperglycemic event based on the data indicative of one or more glucose levels; estimating a plurality of future glucose levels; predicting a total duration for the predicted hyperglycemic event and compare the total duration to a threshold to determine if the predicted hyperglycemic event is a long hyperglycemic event; and if the predicted hyperglycemic event is a long hyperglycemic event, then outputting an alert regarding the long hyperglycemic event.

In some embodiments, the hyperglycemic event is predicted when a glucose level is above the threshold.

In some embodiments, the hyperglycemic event is predicted when a start of a rise in glucose levels is detected.

In some embodiments, the plurality of future glucose levels are estimated based on determined glucose levels from a predetermined time period before the start of the rise in glucose levels of the hyperglycemic event.

In some embodiments, the plurality of future glucose levels are estimated based on determined glucose levels from a predetermined time period before a latest determined glucose level based on the received sensor readings.

In some embodiments, the predetermined time period is about 4 hours. In some embodiments, the total duration for the hyperglycemic event is predicted based on an amount of time the plurality of future glucose levels are above the threshold. In some embodiments, the plurality of future glucose levels comprises a plurality of estimated lower glucose level bounds and a plurality of upper glucose level bounds. In some embodiments, the total duration comprises a lower bound total duration and an upper bound total duration.

In some embodiments, the method further includes the step of determining a risk of hypoglycemia during the long hyperglycemic event; and if a risk of hypoglycemia is determined to exist, then suppress the alert regarding the long hyperglycemic event. In some embodiments, determining the risk of hypoglycemia during the potential long hyperglycemic event comprises determining a glucose pattern, wherein the method further includes the step of: determining an area under the curve (AUC) of the plurality of future glucose levels, wherein the glucose pattern is determined to be one of a plurality of categories based on a comparison of the AUC to a threshold AUC.

In some embodiments, the plurality of categories comprises a low category, a medium category, and a high category.

In some embodiments, the high glucose pattern is categorized based on an area under the curve of the estimated plurality of future glucose levels.

In some embodiments, the method further includes the steps of: determining it the subject has acknowledged the outputted alert; and outputting an additional alert notification after a waiting time if the subject has not acknowledged the outputted alert. In some embodiments, the waiting time is based on the determined total duration for the potential long hyperglycemic event above the high threshold. In some embodiments, the hyperglycemic alert is predicted when a start of a rise in glucose levels is detected, and wherein the method further includes the step of: determining an area under the curve of the plurality of future glucose levels after the start of the rise, and wherein the waiting time is based on the determined area under the curve.

In some embodiments, the waiting time may be varied based on an amount of the determined area under the curve.

In some embodiments, the method further includes the step of: determining a time to output the alert based at least on the determined total duration. In some embodiments, the hyperglycemic event is detected when a start of a rise in glucose levels is detected, and wherein the determined time to output the alert is after a time period after the start of the rise in glucose levels of the potential long hyperglycemic event. In some embodiments, the time period is based on a population dataset. In some embodiments, the time period is based on a dataset of the subject. In some embodiments, the time period is based on a value of a glucose level after the start of the rise in glucose levels is above a threshold value.

In some embodiments, the determined time to output the alert is when a glucose level is equal to or is above the high glucose threshold.

In many embodiments, a method for monitoring a glucose concentration of a user includes the steps of: receiving measurement data related to the glucose concentration of the user; determining a value of the glucose concentration of the user based on the received measurement data; in response to enablement of a high glucose alarm, outputting an alarm when a determined value of the glucose concentration of the user is above a high glucose alarm threshold; and in response to enablement of a long hyperglycemic alert, outputting an alert when estimations based on the received measurement data satisfies at least one condition.

In some embodiments, the high glucose alarm is suppressed if the long hyperglycemic alert is enabled.

In some embodiments, the long hyperglycemic alert is suppressed if the high glucose alarm is enabled.

In some embodiments, method further comprises the steps of: predicting a hyperglycemic event based on the data indicative of one or more glucose levels; predicting a total duration for the predicted hyperglycemic event; and wherein the at least one condition comprises determining if the predicted total duration for the predicted hyperglycemic event is greater than a threshold duration. The at least one condition may be an alert condition as described herein.

In many embodiments, a method for monitoring a glucose concentration of a user includes the steps of: receiving measurement data indicative of one or more glucose levels of the user; predicting a hyperglycemic event based on the data indicative of one or more glucose levels, wherein the step of predicting the hyperglycemic event comprises: determining a start of a rise in glucose levels; and determining if a glucose level after a start of a rise in glucose levels is above a threshold; classifying, by one or more processors, a glucose pattern of the predicted hyperglycemic event as associated with an administered medication dose or not associated with an administered medication dose; and in response to classifying the glucose pattern of the predicted hyperglycemic event as not associated with the administered medication dose, activating an alert when at least one alert condition is met.

In some embodiments, the hyperglycemic event is predicted when a glucose level after the start of the rise in glucose levels is above a high glucose threshold.

In some embodiments, the hyperglycemic event is predicted when the start of a rise in glucose levels is detected.

In some embodiments, the at least one alert condition comprises determining if a plurality of glucose levels after the start of the rise are above the threshold for a period of time. The alert condition may be met if the plurality of glucose levels after the determined start of the rise in glucose levels are above the threshold for a duration greater than a threshold. The threshold may be at least 1 hour, preferably at least 1.5 hours, more preferably at least 2 hours. The alert condition may therefore be met if the hyperglycemic event has a duration of greater than at least 1 hour, preferably at least 1.5 hours, more preferably at least 2 hours such that the hyperglycemic event is a long hyperglycemic event.

In some embodiments, the alert is not activated in response to classifying the glucose pattern of the potential hyperglycemic event as associated with the administered medication dose.

In some embodiments, the method further comprises the steps of: determining if at least one additional glucose level is above the threshold; classifying, by one or more processors, an additional glucose pattern of the predicted hyperglycemic event as associated with an administered medication dose or not associated with an administered medication dose, wherein the predicted hyperglycemic event comprises the start of the rise in glucose levels and the at least one additional calculated glucose level; and in response to classifying the additional glucose pattern of the predicted hyperglycemic event as not associated with the administered medical dose, activating an additional alert when the at least one alert condition is met.

In some embodiments, the glucose pattern of the predicted hyperglycemic event is classified using a predictive model. In some embodiments, inputs of the predictive model comprise time-correlated glucose data. In some embodiments, the time-correlated glucose data is received from a continuous glucose monitor. In some embodiments, inputs of the predictive model comprise medication delivery data. In some embodiments, the medication delivery data comprises manually logged insulin dosing data. In some embodiments, the medication delivery data comprises logged insulin dosing data from a connected medication delivery device.

In some embodiments, inputs of the predictive model comprise data from a population of subjects. In some embodiments, the inputs of the predictive model further comprises data from the user.

In some embodiments, inputs of the predictive model comprise data from the user.

In some embodiments, outputs of the predictive model comprise a probability that the potential hypoglycemic event was associated with an administered medication dose.

In some embodiments, the medication dose is an insulin dose.

In some embodiments, the long hyperglycemic is predicted when a start of a rise in glucose levels is detected, and wherein the administered medical dose was administered within about 30 minutes prior to the start of the rise in glucose levels.

In many embodiments, an analyte monitoring system includes: a sensor control device comprising an analyte sensor, wherein at least a portion of the analyte sensor is configured to be in fluid contact with a bodily fluid of a subject; and a reader device, comprising: wireless communication circuitry configured to receive data indicative of one or more glucose levels; and one or more processors coupled to a memory, the memory storing instructions that, when executed by the one or more processors, cause the one or more processors to: predict a hyperglycemic event based on the data indicative of one or more glucose levels; determine a risk of hypoglycemia after the predicted hyperglycemic event; and if a risk of hypoglycemia is determined to exist, then prevent outputting an alert regarding the predicted hyperglycemic event.

In some embodiments, the instructions further cause the one or more processors to predict a total duration of the hyperglycemic event and compare the total duration to a threshold to determine if the predicted hyperglycemic event is a long hyperglycemic event.

In some embodiments, determining the risk of hypoglycemia during the long hyperglycemic event comprises determining a glucose pattern. In some embodiments, the glucose pattern comprises a plurality of categories. In some embodiments, the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.

In some embodiments, the instructions, when executed by the one or more processors, cause the one or more processors to: determine a probability of the glucose pattern comprising a long high glucose pattern; and determine a time to output the alert based on a comparison of the probability of the glucose pattern comprising the long high glucose pattern to a threshold probability.

In some embodiments, the instructions further cause the one or more processors to: if a risk of hypoglycemia is determined not to exist, then output an alert regarding the predicted hyperglycemic event.

In many embodiments, an analyte monitoring system includes: a sensor control device comprising an analyte sensor, wherein at least a portion of the analyte sensor is configured to be in fluid contact with a bodily fluid of a subject; and a reader device, comprising: wireless communication circuitry configured to receive data indicative of one or more glucose levels; and one or more processors coupled to a memory, the memory storing instructions that, when executed by the one or more processors, cause the one or more processors to: predict a hyperglycemic event based on the data indicative of one or more glucose levels; determine a risk of hypoglycemia after the predicted hyperglycemic event; and if a risk of hypoglycemia is determined to exist, then output an alert regarding the predicted hyperglycemic event and the determined risk of hypoglycemia.

In some embodiments, the instructions further cause the one or more processors to predict a total duration of the hyperglycemic event and compare the total duration to a threshold to determine if the predicted hyperglycemic event is a long hyperglycemic event.

In some embodiments, determining the risk of hypoglycemia during the long hyperglycemic event comprises determining a glucose pattern. In some embodiments, the glucose pattern comprises a plurality of categories. In some embodiments, the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.

In some embodiments, the instructions, when executed by the one or more processors, cause the one or more processors to: determine a probability of the glucose pattern comprising a long high glucose pattern; and determine a time to output the alert based on a comparison of the probability of the glucose pattern comprising the long high glucose pattern to a threshold probability.

In some embodiments, the instructions further cause the one or more processors to: if a risk of hypoglycemia is determined not to exist, then output an alert regarding the predicted hyperglycemic event.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIG. 3 is an illustration of a sequence of events related to an exemplary high glucose alarm.

FIGS. 4A and 4B are flow diagrams depicting example embodiments of methods for outputting a long hyperglycemic event.

FIG. 5A is an illustration of an estimated start of a glucose rise.

FIG. 5B is an illustration of estimating future glucose values and calculating a duration of high glucose values.

FIGS. 5C-5D are illustrations of calculating a duration of high glucose values by accounting for upper and lower bounds associated with the estimated future glucose values.

FIG. 5E is an illustration of calculating upper and lower bounds of duration of high glucose values.

FIG. 6 is an illustration of conditions when a high glucose alarm may be suppressed.

FIG. 7A is an illustration of long high glucose pattern resulting in an alert at the latest time instance.

FIG. 7B is an illustration of conditions that may not trigger a high glucose alarm.

FIG. 8A is a potential embodiment of a long high pre-alert as an option.

FIG. 8B is a potential embodiment of a reminder as an alternative to threshold high glucose alarm.

FIG. 9 is a flow diagram of a modified high alert.

FIG. 10A is a graph of data collected from CGM sensors.

FIG. 10B is a graph of a Receiver Operating Characteristic (ROC) curve.

FIG. 10C is a graph of alert frequency vs. accuracy.

FIGS. 11A-11B are illustrations of calculating AUCs of the estimated future glucose values.

FIG. 12 is an example embodiment of a graph depicting information for determining a hypoglycemia risk and other metrics for a glucose pattern analysis.

FIGS. 13A and 13B are flow diagrams depicting example embodiments of methods for outputting or suppressing notifications related to a hyperglycemic event.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include GUIs, alarms, and digital interfaces for analyte monitoring systems, and systems, methods, and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

As previously described, a number of embodiments described herein provide for improved GUIs, alarms, and digital interfaces for analyte monitoring systems, wherein the alarms and GUIs are actionable, user-friendly, and provide for rapid access to physiological information of a user. According to some embodiments, methods, systems, and interfaces relating to determining long, high glucose alerts or pre-alerts are described. In other embodiments, methods, systems, and interfaces for determining high glucose alarms, including determining if a glucose pattern of a potential hyperglycemic event is associated with an administered medication dose or not are described. In still other embodiments, methods, systems, and interfaces relating to options for enabling and disabling high glucose alarms and long, high glucose alerts or pre-alerts under different conditions are described.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

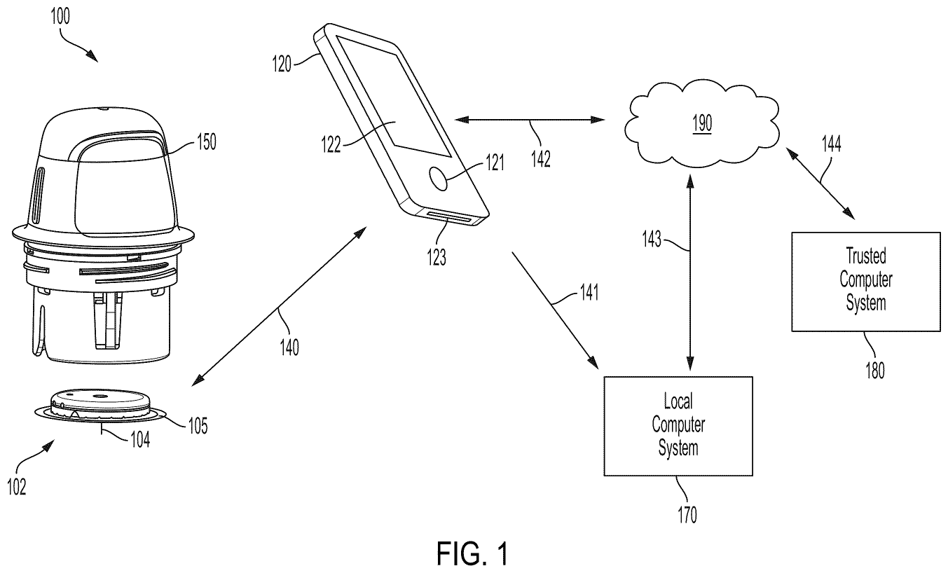

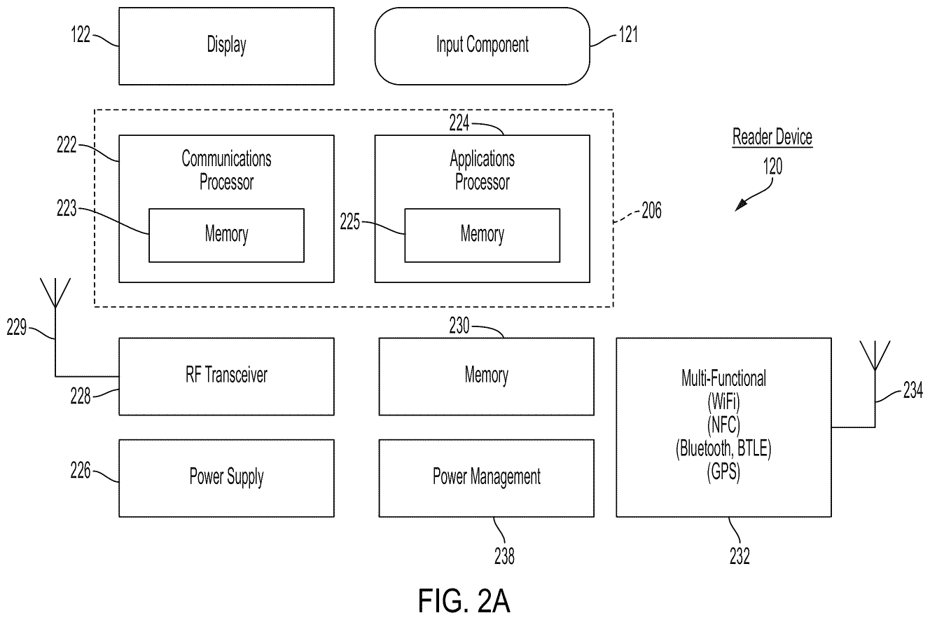

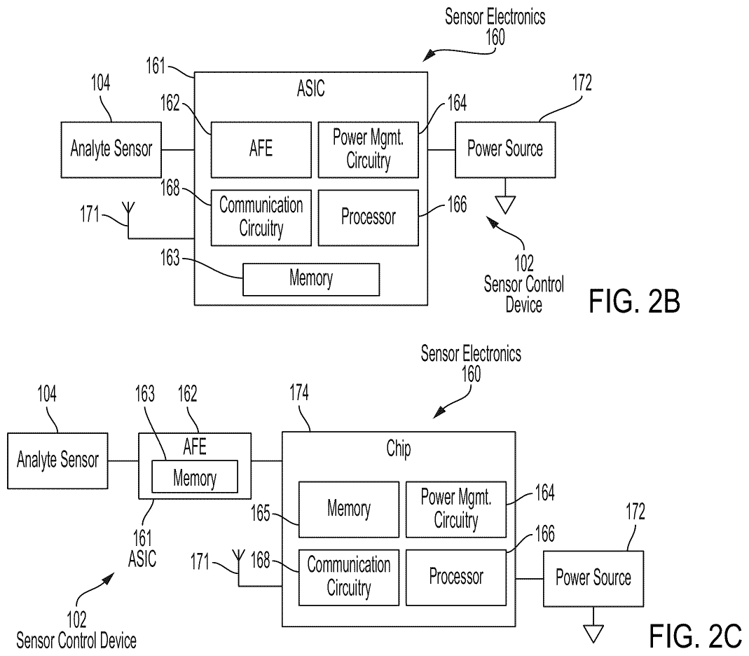

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, data acquired from sensor control device 102 can be stored on reader device 120. According to one aspect of some embodiments, such data can include the model number and serial number of sensor control device 102, as well as information relating to the sensor control device 102's status, market code, or network address. In some embodiments, such data can also include error events detected by sensor control device 102. In addition, in some embodiments, cither or both of current glucose values and historical glucose values can include one or more time stamps (e.g., factory time, UTC time, user's local time based on time zone, and the current time zone).

In some embodiments, sensor control device 102 can store data such that if reader device 120 is not in communication with sensor control device 102 (e.g., if reader device 120 is out of a wireless communication range, is powered off, or is otherwise unable to communicate with sensor control device 102), when reader device 120 re-establishes communication with sensor control device 102, data can then be backfilled to reader device 120. According to some embodiments, data that can be backfilled can include, but is not limited to, current and historical glucose values, as well as error events. Further details regarding data backfilling can be found in U.S. Pat. No. 10,820,842, as well as U.S. Publ. No. 2021/0282672 (“the '672 Publication”), both of which are hereby incorporated by reference in their entireties for all purposes.

According to some embodiments, each current glucose value and/or historical glucose value acquired from sensor control device 102 can further be validated on reader device 120, such as, for example, by performing a CRC integrity check to ensure that the data has been transferred accurately. In some embodiments, for example, a data quality mask of the current glucose value and/or historical glucose value can be checked to ensure that the reading is correct and can be displayed as a valid reading on the reader device 120.

According to another aspect of some embodiments, reader device 120 can include a database for storing any or all of the aforementioned data. In some embodiments, the database can be configured to retain data for a predetermined period of time (e.g., 30 days, 60 days, 90 days, six months, one year, etc.). According to some embodiments, the database can be configured to delete data after it has been uploaded to a cloud server. In other embodiments, database can be configured for a clinical setting, in which data is retained for a longer period of time (e.g., one year) relative to a non-clinical setting. In addition to the aforementioned data (e.g., current and/or historical glucose values, error events, etc.), the database on reader device 120 can also store user configuration information (e.g., login ID, notification settings, regional settings, and other preferences), as well as application configuration information (e.g., cloud settings, URLs for uploading data and/or error events, version information, etc.). The database can be encrypted to prevent a user from inspecting the data content directly even if the operating system of reader device 120 is compromised.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### High Glucose Alarms or Alerts

Various example embodiments relating to alarming and alarm suppression methods, alarm interfaces, alarm setup interfaces, alerting and alert suppression methods, alert interfaces, alert setup interfaces, and other related features will now be described. It will be understood by those of skill in the art that any one or more of the example embodiments of the methods, interfaces, and systems described herein can either be implemented independently, or in combination with any of the other embodiments described in the present application.

### Example Embodiments of Hyperglycemic Alarms

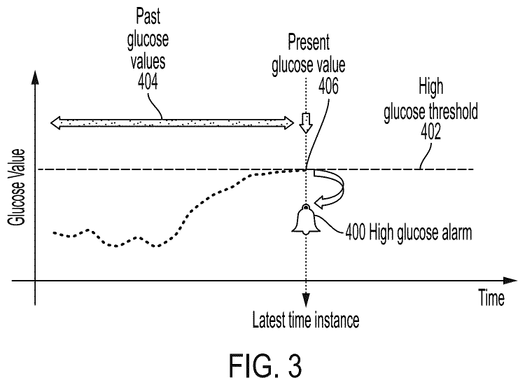

Example embodiments of high glucose alarms will now be described. According to one aspect of the embodiments, high glucose alarms will present an alarm, alert, or notification to the user when his or her glucose level has risen above a high glucose threshold 402 (e.g., above 180 mg/dL). As seen in FIG. 3, when glucose levels cross the high threshold, a high glucose alarm 400 is enunciated. In many embodiments, the high glucose threshold levels 402 may be adjustable.

### Example Embodiments of Long Hyperglycemic Alerts

Various example embodiments relating to alarming and alarm suppression methods, alarm interfaces, alarm setup interfaces, and other related features will now be described. It will be understood by those of skill in the art that any one or more of the example embodiments of the methods, interfaces, and systems described herein can either be implemented independently, or in combination with any of the other embodiments described in the present application.

There are numerous advantages of long hyperglycemia alarms. First, being able to know that a post-meal high glucose will persist before the event has taken place gives the user the opportunity to take a post-meal correction bolus closer to the start of the meal, when the pharmacokinetics/pharmacodynamics (PK-PD) is better matched. This translates to better glycemic improvement whenever the hassle of an additional dose is suggested, leading to better trust in the relevance of the notifications or alerts. From a meal choice point of view, it can be useful feedback when deciding to get a second helping. This feedback may also be useful when deciding the timing of exercise, because exercise can lower post-meal glucose when timed correctly.

Second, such a predictive alarm for long hyperglycemia may also prevent alarm fatigue from irrelevant alarms from each initial post-meal spike. Using machine learning tools, a pre-alert is created to only notify when the glucose rise is predicted to persist long enough to warrant action. The proposed pre-alert increases the clinical relevance of each notification, thereby increasing the user's trust of the CGM system. Overall, this increases the trust of the user that the system provides precision advice.

Third, categorizing the post-meal high glucose, and not alerting when the post-meal is not likely to persist without further intervention, also prevents rebound hypoglycemia from “rage blousing.”

Real-world patient data of our CGM system may be used to define the development and evaluation datasets. Persistent high glucose (e.g., >2 hrs & >180 mg/dL) segments may be defined as dataset truth. Machine Learning (ML) techniques, such as Random Forest and Logistic Regression, may be used to develop a pre-alert detector, which may be based solely on the user's CGM history or may be based on a training dataset.

HCPs commonly suggest first time CGM users to disable the high glucose alert, and then start from a relatively high threshold to gradually lower the threshold to arrive at a practical compromise between picking up long high glucose versus unnecessary distraction from short ones. The long, high glucose alert or pre-alert improves upon the standard high alert by reducing nuisance alerts while increasing the lead time to act in cases that need user intervention, thereby increasing the user's trust of the CGM system. Trust in the system maximizes user retention and sustained clinical benefit of using a CGM system.

Example embodiments of long hyperglycemic alerts will now be described. In a general sense, long hyperglycemic alerts for analyte monitoring systems may present an alert or a pre-alert to the user when estimated glucose levels are determined to satisfy at least one alert condition.

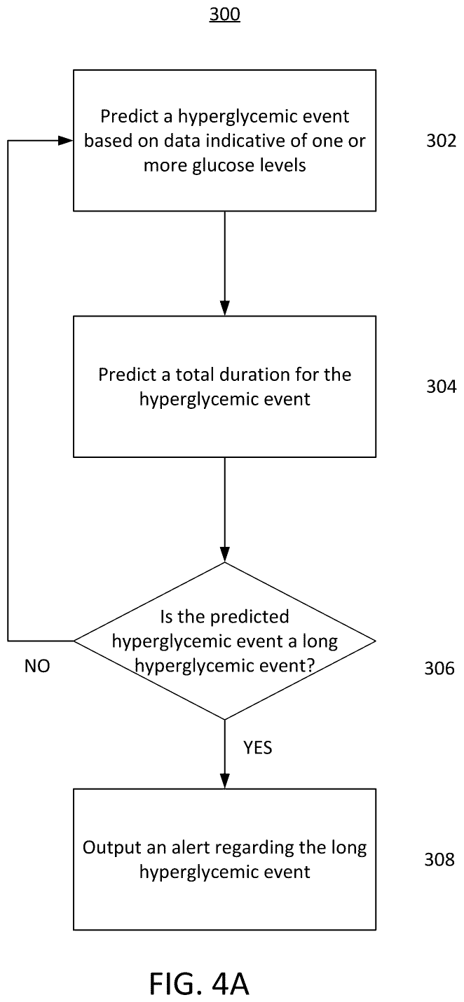

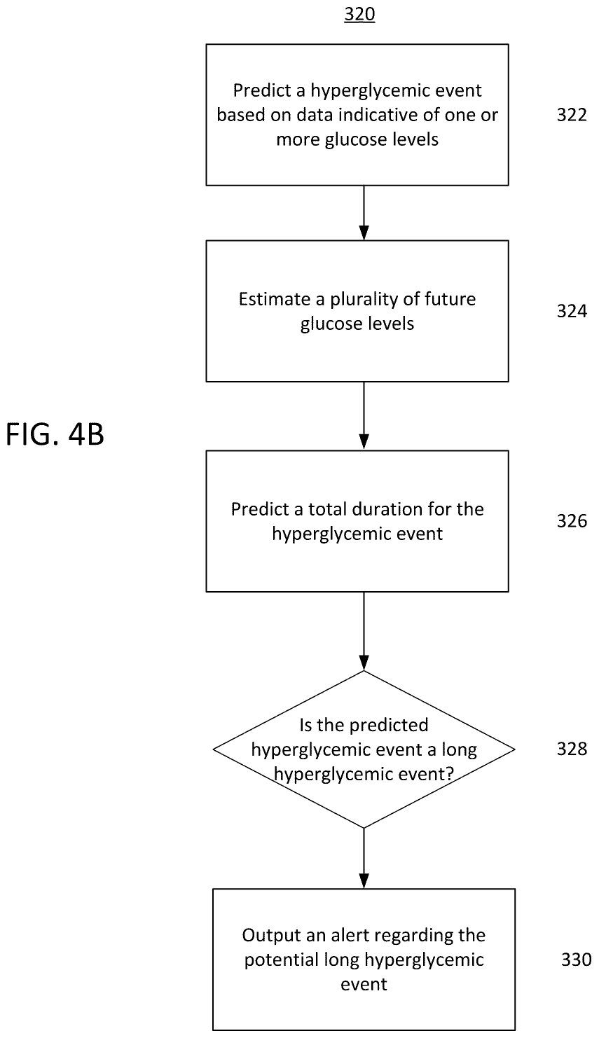

FIG. 4A depicts an example embodiment of a method 300 for outputting a long hyperglycemic alert. At Step 302, a hyperglycemic event is predicted based on data indicative of one or more glucose levels.

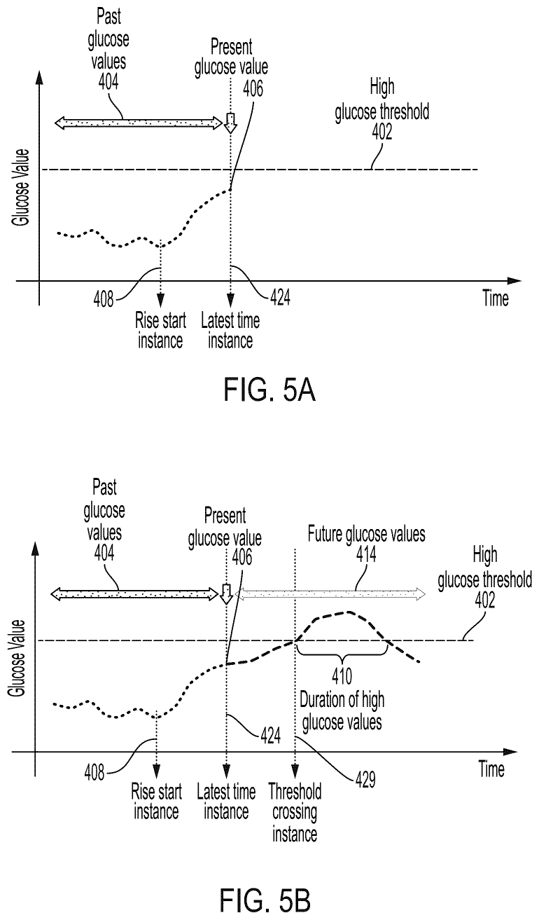

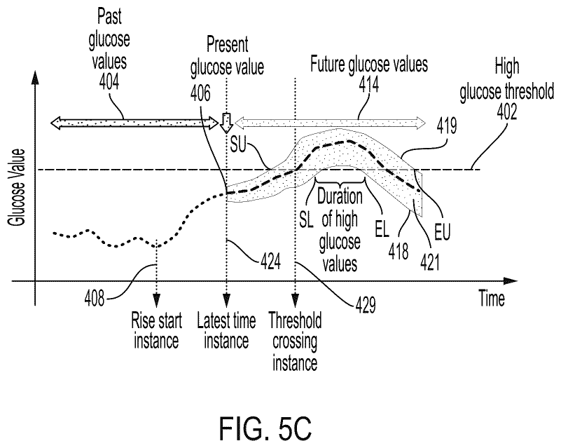

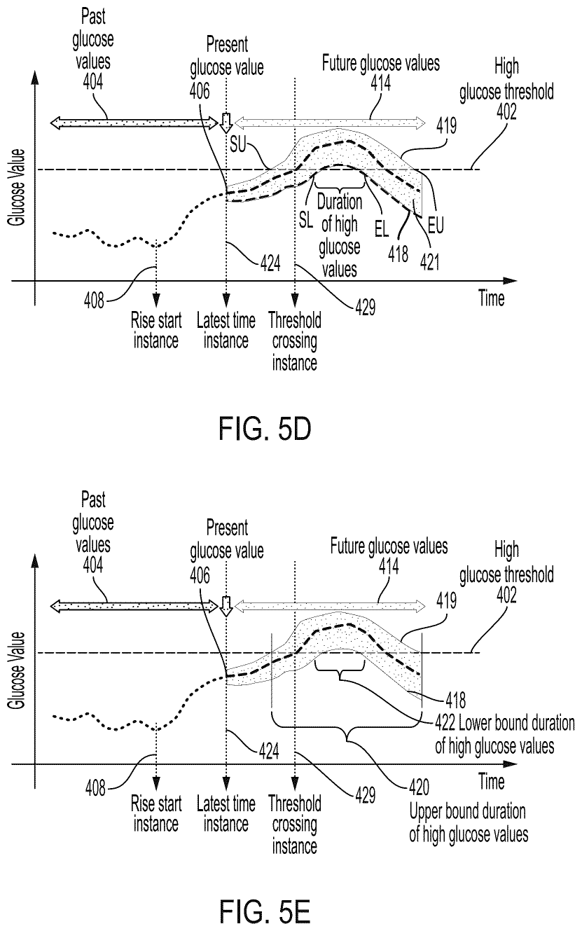

In some embodiments, the hyperglycemic event may be predicted by detecting a start of a rise in glucose levels. In some embodiments, a start of a rise in glucose levels may be detected based on a calculated rate of change or glucose levels within a time period, one or more glucose levels over a threshold level, or a combination thereof. In some embodiments, past and present glucose values 404, 406 from a glucose monitoring system may be used as input to a glucose rise estimate function. The function may estimate the latest start of a glucose rise, denoted by “Rise start instance” 408 in FIG. 5A. In some embodiments, rise start instances 408 that are already higher than a high glucose threshold 402 may be considered differently or excluded from later steps of estimation of total duration of high glucose. Once the subsequent glucose values are deemed to return to a baseline value or the long hyperglycemic event is determined to have ended, the function can be used to estimate the next latest start again.

Examples of methods for marking the start of a glucose rise are the estimation of meal start are described in US 2017/0185748, the Glucose Rate Increase Detector (GRID) method as described in Harvey, R. A. et al. “Design of the Glucose Rate Increase Detector,” J Diabetes Sci Technol. 8 (2): 307-320 (March 2014), the Variable State Dimension (VSD) method as described in Sabatini, A. M., “Variable-State-Dimension Kalman-based Filter for orientation determination using inertial and magnetic sensors,” Sensors (Basel) 12 (7): 8491-506 (2012), and methods that utilize both glucose and insulin data described in Samadi, S. et al., “Meal Detection and Carbohydrate Estimation Using Continuous Glucose Sensor Data,” IEEE J Biomed Health Inform. 21 (3): 619-627 (May 2017), all of which are hereby expressly incorporated by reference in their entireties for all purposes. In some embodiments, if a mealtime is known, it may be used as an alternative for marking the start of estimation. The delay of meal absorption, however, causes the start of a glucose rise to have a detection lag compared with actual mealtime. Although both can be marked as the start, a time shift for model inputs may be required when the same model is used for these two different cases.

In some embodiments, the hyperglycemic event may be predicted when a measured glucose level is detected that is at or above a high glucose threshold. See, e.g., threshold crossing instance 429 of FIG. 5B.

At Step 304, a total duration for the hyperglycemic event is predicted.

In some embodiments, the total duration 410 of the hyperglycemic event may be determined based on using all available glucose values up to the present to calculate an estimated future glucose. In some embodiments, recent glucose values from a time period (e.g., about 5 hours, alternatively about 4 hours, alternatively about 3 hours) prior to crossing a threshold value. The available glucose values since the latest start of a glucose rise 408 and estimated future glucose levels may then be used to calculate the total duration of high glucose. Estimation of future glucose value may be calculated using a variety of glucose prediction methods. As shown in FIG. 5B, the total duration 410 may be determined based on an amount of time that the estimated or future glucose levels 414 are above a threshold value 402. The threshold value may be at least 140 mg/L, alternatively at least 160 mg/L, alternatively at least 180 mg/L, alternatively at least 200 mg/L, alternatively at least 220 mg/dL, alternatively at least 240 mg/dl, alternatively at least 260 mg/dL. The window of time in which the estimated future glucose values are determined may be a fixed duration relative to the rise start instance 408, or a fixed duration relative to the latest time instance 424. In some embodiments, the total duration 410 for the potential long hyperglycemic event may be determined by applying different thresholds for the start and end of the event. For example, the threshold value 402 may be used to determine the start of the hyperglycemic event, and a second threshold value may be used to determine the end of the hyperglycemic event. The time elapsed between the start and end is then defined as the duration of the event. In some embodiments, the second threshold value used to determine the end of the hyperglycemic event may be lower than the threshold value used to determine the start of the hyperglycemic event.

In some embodiments, the total duration 410 for the potential long hyperglycemic event may be determined by estimating upper and lower bounds 419, 418 associated with the estimated future glucose values 414, as shown in FIG. 5D. With the upper and lower bounds 419, 418, the start of high glucose can be at points marked SU or SL, respectively. Similarly, the end of high glucose can be at points marked EU or EL.

In some embodiments, the lower bounds 418 (the lower red dashed line representing the lower bound of future glucose values) may be used to mark the start and end. Then, available glucose values since the latest start of a glucose rise 408 and the lower bounds of the estimated future glucose values are used to determine the earliest time a high glucose threshold is exceeded. In addition, available glucose values since the latest start of a glucose rise 408 and the lower bounds of the estimated future glucose values may be used to determine the earliest time glucose will return below the high glucose threshold. The time difference between these two may be defined as the total duration of high glucose.

In other embodiments, as seen in FIG. 5E, instead of determining a single estimate for the total duration of high glucose at a given time, a spread of future glucose values 421 with upper and lower bounds 419, 418 may be used to calculate upper and lower bounds of the estimated total duration of high glucose 420, 422. For example, the earliest time that the lower bound of the estimated glucose values return below the high glucose threshold may be used to determine the lower bound 422 of the estimated total duration of high glucose. The upper bound duration of high glucose values 420 may be a duration of high glucose values based on upper bound estimates of future glucose values. The upper limit 419 of the spread of estimated glucose values in FIG. 5E crosses the high glucose threshold 402 at the edges of the upper bound duration of high glucose values 420. Similarly, the earliest time that the upper bound of the estimated glucose values return below the high glucose threshold may be used to determine the upper bound 420 of the estimated total duration of high glucose.

In other embodiments, estimation of upper and lower bounds of the estimated total duration 420, 422 of high glucose may be directly calculated using one of regression Machine learning methods, without calculating the upper and lower bounds for estimated future glucose. The inputs for the estimation of the total duration of high glucose may be directly calculated using a Machine learning method, without calculating the upper and lower bounds for estimated future glucose.

The inputs for estimation of high glucose duration can at least be classified into two groups. First, inputs may include the short-term and transitory trend or pattern of one glucose rising excursion. For example, a time series of four glucose values after rise start instance 408 may be input for estimating high glucose duration. Second, inputs may include metrics or statistics of the user's history of glucose data received, such as average, minimum, maximum, standard deviation over a relatively short history like last 24 hours or last bedtime or long history like the last two weeks. In addition to specifying the duration to define history, another aspect may be whether the statistic defined absolute values as relative to the normal values for the subject to capture patient specificity. Moreover, in addition to glucose, other inputs such as insulin, meal content/amount may be used to estimate high glucose duration.

The inputs for estimation of high glucose duration can be based on various glucose metrics. In some embodiments, the glucose metrics may be used to determine how to count the total duration when the glucose time series values (from the estimated future glucose values) goes below and above the threshold several times in recent succession. In other embodiments, the estimated future glucose values may be calculated with upper and lower bounds, and some traces go above and below the threshold several times. A plurality of glucose metrics could help with this determination. For example, one metric may be when glucose values within the total duration of the predicted hyperglycemic event are predicted to have a Time Above Range exceeding 250 mg/dL (TAR250) for at least 30 minutes. In other embodiments, a metric may be whether or not recent similar excursions lasted longer than a predetermined period of time. If recent similar excursions did not, then the metric may reduce the estimated total duration by a scaling factor greater than 0 but less than 1, or reduce the total duration by a pre-determined equation as a function of that metric. The metrics may be determined from glucose levels received and/or predicted. For example, given the timing of the prediction at or slightly before crossing the high glucose threshold, then only glucose levels predicted are involved because none of the recent received glucose levels exceed that threshold. In another example, a metric reflecting glucose rate of change in the past 2 hours may rely solely on the received glucose levels. In another example, a similar metric reflecting a similar 2-hour glucose rate of change until the first crossing of the threshold can include both received glucose and predicted glucose values.

At Step 306, it is determined if the predicted hyperglycemic event is a long hyperglycemic event. In some embodiments, this determination may be based on a comparison of the total duration to a threshold duration. If the total duration is above the threshold duration, then the predicted hyperglycemic event may be classified as a long hyperglycemic event. The threshold duration may be at least 1 hour, alternatively at least 1.5 hours, alternatively at least 2 hours, alternatively at least 2.5 hours, alternatively at least 3 hours, alternatively at least 3.5 hours, alternatively at least 4 hours.

At Step 308, an alert 430 regarding the long hyperglycemic event is outputted. The alert may be outputted as an alarm, a notification, a sound (or vibration), and display a visual notification on the display of reader device 120 (e.g., on a lock screen), even if reader device 120 is muted or configured in a “Do Not Disturb” mode.

FIG. 4B depicts an example embodiment of a method 320 for outputting a long hyperglycemic alert. At Step 322, as explained with respect to method 300, a hyperglycemic event is predicted.

In some embodiments, the hyperglycemic event may be predicted by detecting a start of a rise in glucose levels. Past and present glucose values 404, 406 from a glucose monitoring system may be used as input to a glucose rise estimate function. The function may estimate the latest start of a glucose rise, denoted by “Rise start instance” 408 in FIG. 5A. In some embodiments, rise start instances 408 that are already higher than a high glucose threshold 402 may be considered differently or excluded from later steps of estimation of total duration of high glucose. Once the subsequent glucose values are deemed to return to a baseline value or the long hyperglycemic event is determined to have ended, the function can be used to estimate the next latest start again. Examples of methods for marking the start of a glucose rise are the estimation of meal start were described elsewhere in this application.

In some embodiments, the potential long hyperglycemic event may be detected when a glucose level is detected that is at or above a high glucose threshold.

At Step 324, a plurality of future glucose levels 414 after the start of the rise 408 may be estimated. Estimation of future glucose values 414 can be done using a variety of glucose prediction methods, such as those described in Oviedo, S. et al. “A review of personalized blood glucose prediction strategies for TIDM patients.” International journal for numerical methods in biomedical engineering 33, no. 6 (2017): e2833; Simone, F. et al. “Linear Model Identification for Personalized Prediction and Control in Diabetes,” IEEE Transactions on Biomedical Engineering, vol. 69, pp. 558-568, 2022; Sevil, M. et al. “Physical activity and psychological stress detection and assessment of their effects on glucose concentration predictions in diabetes management.” IEEE Transactions on Biomedical Engineering 68, no. 7 (2021): 2251-2260; and Li, N. et al. “Prediction of blood glucose concentration for type 1 diabetes based on echo state networks embedded with incremental learning,” Neurocomputing, 2019 Oct. 23/online 23 Oct. 2019, all of which are hereby expressly incorporated by reference in their entireties for all purposes. The window used to estimate future glucose values 414 can be a fixed duration relative to the rise start instance, or a fixed duration relative to the latest time instance. In some embodiments, all available glucose values up to the present to calculate a plurality of estimated future glucose values. In some embodiments, recent glucose values from a time period (e.g., about 5 hours, alternatively about 4 hours, alternatively about 3 hours prior to crossing a threshold value) may be used to calculate a plurality of estimated future glucose values.

At Step 326, a total duration 410 for the hyperglycemic event may be predicted.

In some embodiments, as shown in FIG. 5B, the total duration 410 may be predicted based on an amount of time that the estimated or future glucose levels 414 are above a threshold value. The window of time in which the estimated future glucose values 414 are estimated may be a fixed duration relative to the rise start instance 408, or a fixed duration relative to the latest time instance 424.

In some embodiments, the total duration 410 for the hyperglycemic event may be determined by estimating upper and lower bounds 419, 418 associated with a spread of estimated future glucose values 421, as shown in FIG. 5C. As seen in FIG. 5D, with the upper and lower bounds 419, 418, the start of high glucose can be at points marked SU or SL, respectively. Similarly, the end of high glucose can be at points marked EU or EL.

In some embodiments, the lower bounds 418 (the lower red dashed line representing the lower bound of future glucose values in FIG. 5D) may be used to mark the start and end. Then, available glucose values since the latest start of a glucose rise 408 and the lower bounds of the estimated future glucose values 418 are used to determine the earliest time a high glucose threshold is exceeded. In addition, available glucose values since the latest start of a glucose rise 408 and the lower bounds 418 of the estimated future glucose values may be used to determine the earliest time glucose will return below the high glucose threshold. The time difference between these two may be defined as the total duration of high glucose.

In some embodiments, the upper bounds 419 may be used to mark the start and end. Then, available glucose values since the latest start of a glucose rise 408 and the upper bounds 419 of the estimated future glucose values are used to determine the earliest time a high glucose threshold is exceeded. In addition, available glucose values since the latest start of a glucose rise 408 and the upper bounds 419 of the estimated future glucose values may be used to determine the earliest time glucose will return below the high glucose threshold. The time difference between these two may be defined as the total duration of high glucose.

In some embodiments, the upper bounds 419 may be used to mark the start and the lower bounds 418 may be used to mark the end. Then, available glucose values since the latest start of a glucose rise 408 and the upper bounds of the estimated future glucose values 419 are used to determine the earliest time a high glucose threshold is exceeded. In addition, available glucose values since the latest start of a glucose rise 408 and the lower bounds 418 of the estimated future glucose values may be used to determine the earliest time glucose will return below the high glucose threshold. The time difference between these two may be defined as the total duration of high glucose.

In some embodiments, the lower bounds 418 may be used to mark the start and the upper bounds 419 may be used to mark the end. Then, available glucose values since the latest start of a glucose rise 408 and the lower bounds of the estimated future glucose values 418 are used to determine the earliest time a high glucose threshold is exceeded. In addition, available glucose values since the latest start of a glucose rise 408 and the upper bounds 419 of the estimated future glucose values may be used to determine the earliest time glucose will return below the high glucose threshold. The time difference between these two may be defined as the total duration of high glucose.

In other embodiments, as seen in FIGS. 5C-5E, instead of determining a single estimate for the total duration of high glucose at a given time, the upper and lower bounds of the estimated glucose values 419, 418 may be used to calculate upper and lower bounds of the estimated total duration of high glucose 420, 422. For example, the earliest time that the lower bound of the estimated glucose values return below the high glucose threshold 402 may be used to determine the lower bound of the estimated total duration of high glucose. The upper bound duration of high glucose values 420 may be a duration of high glucose values based on upper bound estimates of future glucose values. The upper limit 419 of the spread of future glucose values 421 in FIG. 5E crosses the high glucose threshold 402 at the edges of the upper bound duration of high glucose values 420. As explained with respect to other embodiments, different combinations of the upper and lower bounds 419, 418 may be used to determine the duration. For example, the upper bounds 416 may be used to determine both the start and finish of the total duration. Alternatively, the lower bounds 418 may be used to determine both the start and finish of the total duration. Alternatively, the upper bounds 419 may be used to determine the start and the lower bounds 418 may be used to determine the end of the total duration. Alternatively, the lower bounds 418 may be used to determine the start and the upper bounds 419 may be used to determine the end of the total duration.

In other embodiments, estimation of upper and lower bounds of the estimated total duration of high glucose can be directly calculated using one of regression Machine learning methods, without calculating the upper and lower bounds for estimated future glucose. The inputs for the estimation of the total duration of high glucose may be directly calculated using one of Machine learning methods, without calculating the upper and lower bounds for estimated future glucose.

The inputs for estimation of high glucose duration can at least be classified into two groups. First, inputs describing the short-term and transitory trend of one glucose rising excursion. For example, a time series of four glucose values after rise start instance can be input for estimating high glucose duration. Second, inputs describing metrics or statistics of the user's history of glucose data, such as average, minimum, maximum, standard deviation over a relatively short history like last 24 hours or last bedtime or long history like the last two weeks. In addition to specifying the duration to define history, the other term of freedom is whether the statistic defined absolute values as relative to the normal values for the subject to capture patient specificity. Moreover, in addition to glucose, other inputs such as insulin, meal content/amount may be used to estimate high glucose duration.

At Step 328, it is determined if the predicted hyperglycemic event is a long hyperglycemic event. In some embodiments, this determination may be based on a comparison of the total duration to a threshold duration. If the total duration is above the threshold duration, then the predicted hyperglycemic event may be classified as a long hyperglycemic event. The threshold duration may be at least 1 hour, alternatively at least 1.5 hours, alternatively at least 2 hours, alternatively at least 2.5 hours, alternatively at least 3 hours, alternatively at least 3.5 hours, alternatively at least 4 hours.

At Step 330, an alert 430 regarding the potential long hyperglycemic event is outputted. The alert 430 may be outputted as an alarm, a notification, a sound (or vibration), and display a visual notification on the display of reader device 120 (e.g., on a lock screen), even if reader device 120 is muted or configured in a “Do Not Disturb” mode.

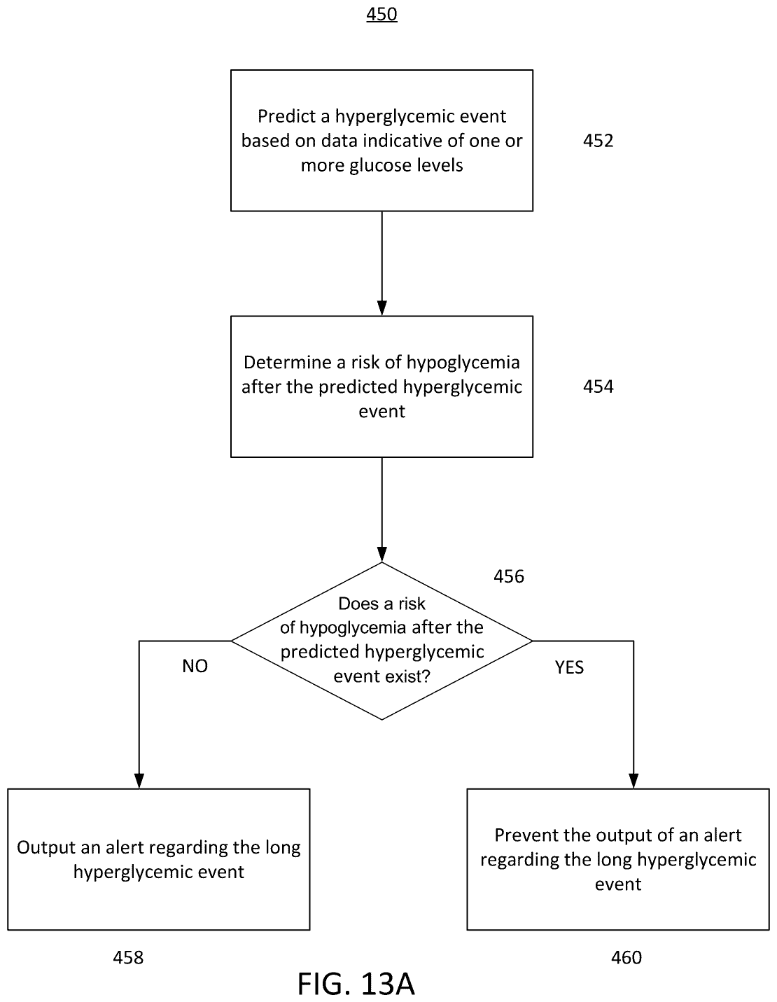

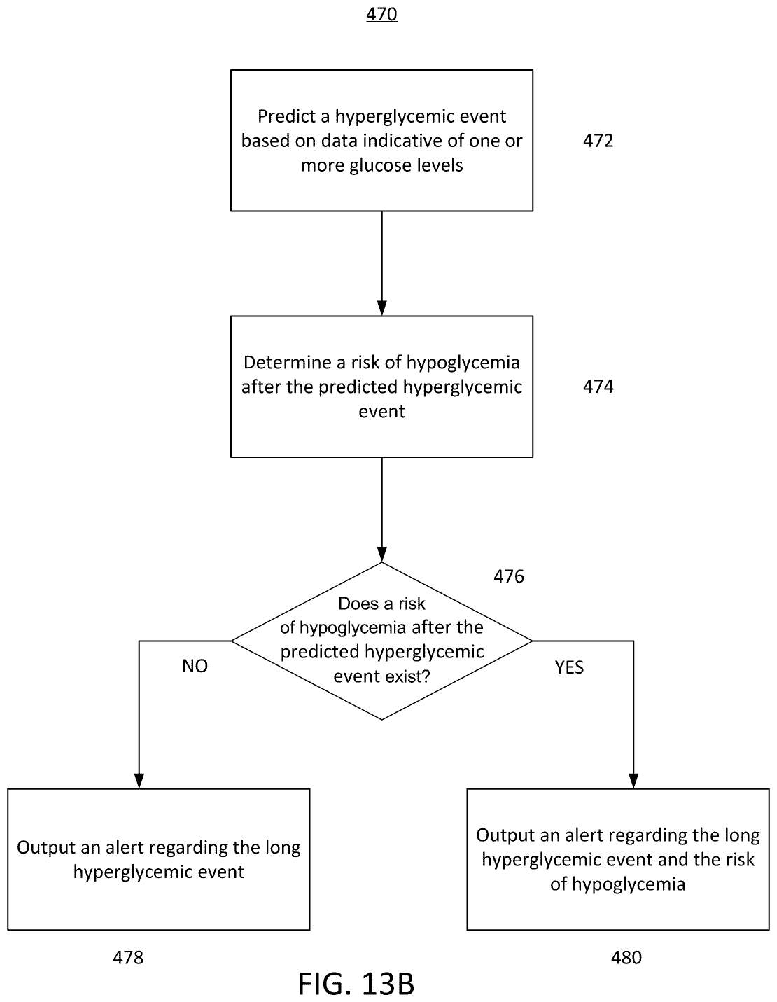

FIG. 13A depicts an example embodiment of a method 450 for preventing or suppressing a long hyperglycemic alert if a risk of hyperglycemia is detected. At Step 452, a hyperglycemic event is predicted based on data indicative of one or more glucose levels.

In some embodiments, the hyperglycemic event may be predicted by detecting a start of a rise in glucose levels. In some embodiments, a start of a rise in glucose levels may be detected based on a calculated rate of change or glucose levels within a time period, one or more glucose levels over a threshold level, or a combination thereof. In some embodiments, past and present glucose values 404, 406 from a glucose monitoring system may be used as input to a glucose rise estimate function. The function may estimate the latest start of a glucose rise, denoted by “Rise start instance” 408 in FIG. 5A. In some embodiments, rise start instances 408 that are already higher than a high glucose threshold 402 may be considered differently or excluded from later steps of estimation of total duration of high glucose. Once the subsequent glucose values are deemed to return to a baseline value or the long hyperglycemic event is determined to have ended, the function can be used to estimate the next latest start again.

Examples of methods for marking the start of a glucose rise are the estimation of meal start are described in US 2017/0185748, the Glucose Rate Increase Detector (GRID) method as described in Harvey, R. A. et al. “Design of the Glucose Rate Increase Detector,” J Diabetes Sci Technol. 8 (2): 307-320 (March 2014), the Variable State Dimension (VSD) method as described in Sabatini, A. M., “Variable-State-Dimension Kalman-based Filter for orientation determination using inertial and magnetic sensors,” Sensors (Basel) 12 (7): 8491-506 (2012), and methods that utilize both glucose and insulin data described in Samadi, S. et al., “Meal Detection and Carbohydrate Estimation Using Continuous Glucose Sensor Data,” IEEE J Biomed Health Inform. 21 (3): 619-627 (May 2017), all of which are hereby expressly incorporated by reference in their entireties for all purposes. In some embodiments, if a mealtime is known, it may be used as an alternative for marking the start of estimation. The delay of meal absorption, however, causes the start of a glucose rise to have a detection lag compared with actual mealtime. Although both can be marked as the start, a time shift for model inputs may be required when the same model is used for these two different cases.

In some embodiments, the hyperglycemic event may be predicted when a measured glucose level is detected that is at or above a high glucose threshold. See, e.g., threshold crossing instance 429 of FIG. 5B.

At step 454, a risk of hypoglycemia after the predicted hyperglycemic event is determined.

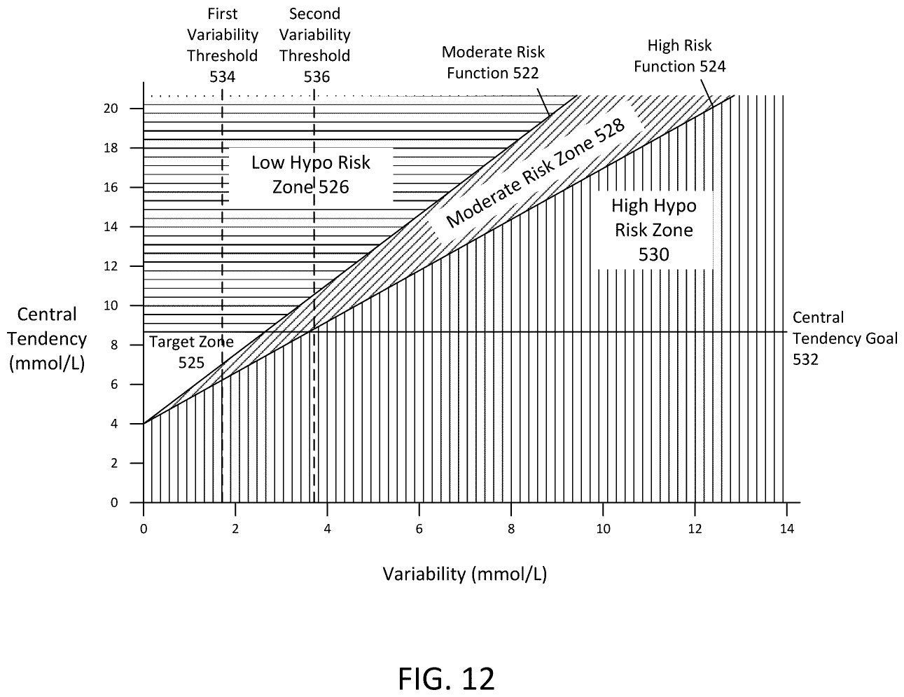

One such methodology for determining a risk of hypoglycemia is described with respect to FIG. 12, showing an example embodiment of a framework for determining hypo risk and other metrics. While FIG. 12 is intended to convey the framework to the reader, however, this framework can be implemented electronically in numerous different ways, such as with a software algorithm (e.g., a mathematical formula, a set of if-else statements, etc.), a lookup table, firmware, a combination thereof, or otherwise.

FIG. 12 is a graph of central tendency versus variability (e.g., low range variability) that can be used to evaluate or identify a region or zone that holds or corresponds to a determined central tendency and variability data pair for a particular time of day (TOD). Any number of two or more zones can be used. In this embodiment the data pair can correspond to a target zone 525 or one of three hypo risk zones: a low zone 526, a moderate zone 528, or a high zone 530. A first hypo risk function (e.g., a curved or linear boundary), referred to as moderate risk function 522, differentiates between low zone 526 and moderate zone 528. A second hypo risk function, referred to as high risk function 524, differentiates between moderate zone 528 and high zone 530. The central tendency and variability data pair can be evaluated against or compared to the zones to determine a hypo risk metric for the corresponding TOD period.

The hypo risk functions 522 and 524 can be implemented in the DGA explicitly as a mathematical function (e.g., a polynomial) or can be implemented implicitly, such as by defining each zone by the pairs it contains, use of a lookup table, set of if-else statements, threshold comparisons, or otherwise. The hypo risk functions 522 and 524 can be preloaded into a software application, or can be downloaded from trusted computer system 580, or can be set by another party such as the HCP. Once implemented, the hypo risk functions 522 and 524 can be treated as fixed or can be adjusted by the user or HCP. Example methodologies for determining the hypo risk function are described in U.S. Patent Publ. No. 2018/0188400 (the '400 publication), which is incorporated by reference herein for all purposes.

At step 456, if a risk of hypoglycemia exists, then in step 460, the processor prevents the output of an alert regarding the long hyperglycemic event.

At step 456, if a risk of hypoglycemia does not exist, then in step 458, the processor outputs an alert regarding the long hyperglycemic event.

Avoiding or suppressing the long hyperglycemic alert notification is beneficial when a person's combination of diabetes state (e.g., reduced glucagon response to hypoglycemia) and treatment modality (e.g., using insulin pen and/or very busy schedule) as indicated by the recent history of glycemic control has a high risk of hypoglycemia after hyperglycemia. Avoiding or suppressing the notification can prevent actions (e.g., administering too much correction dose at less-than-ideal times) that can lead to hypoglycemic risk.

FIG. 13B depicts an example embodiment of a method 470 for outputting a long hyperglycemic alert and a hypoglycemia alert if a risk of hyperglycemia is detected. At Step 472, a hyperglycemic event is predicted based on data indicative of one or more glucose levels.

In some embodiments, the hyperglycemic event may be predicted by detecting a start of a rise in glucose levels. In some embodiments, a start of a rise in glucose levels may be detected based on a calculated rate of change or glucose levels within a time period, one or more glucose levels over a threshold level, or a combination thereof. In some embodiments, past and present glucose values 404, 406 from a glucose monitoring system may be used as input to a glucose rise estimate function. The function may estimate the latest start of a glucose rise, denoted by “Rise start instance” 408 in FIG. 5A. In some embodiments, rise start instances 408 that are already higher than a high glucose threshold 402 may be considered differently or excluded from later steps of estimation of total duration of high glucose. Once the subsequent glucose values are deemed to return to a baseline value or the long hyperglycemic event is determined to have ended, the function can be used to estimate the next latest start again.

Examples of methods for marking the start of a glucose rise are the estimation of meal start are described in US 2017/0185748, the Glucose Rate Increase Detector (GRID) method as described in Harvey, R. A. et al. “Design of the Glucose Rate Increase Detector,” J Diabetes Sci Technol. 8 (2): 307-320 (March 2014), the Variable State Dimension (VSD) method as described in Sabatini, A. M., “Variable-State-Dimension Kalman-based Filter for orientation determination using inertial and magnetic sensors,” Sensors (Basel) 12 (7): 8491-506 (2012), and methods that utilize both glucose and insulin data described in Samadi, S. et al., “Meal Detection and Carbohydrate Estimation Using Continuous Glucose Sensor Data,” IEEE J Biomed Health Inform. 21 (3): 619-627 (May 2017), all of which are hereby expressly incorporated by reference in their entireties for all purposes. In some embodiments, if a mealtime is known, it may be used as an alternative for marking the start of estimation. The delay of meal absorption, however, causes the start of a glucose rise to have a detection lag compared with actual mealtime. Although both can be marked as the start, a time shift for model inputs may be required when the same model is used for these two different cases.

In some embodiments, the hyperglycemic event may be predicted when a measured glucose level is detected that is at or above a high glucose threshold. Sec, e.g., threshold crossing instance 429 of FIG. 5B.

At step 474, a risk of hypoglycemia after the predicted hyperglycemic event is determined.

One such methodology for determining a risk of hypoglycemia is described with respect to FIG. 12, showing an example embodiment of a framework for determining hypo risk and other metrics. While FIG. 12 is intended to convey the framework to the reader, however, this framework can be implemented electronically in numerous different ways, such as with a software algorithm (e.g., a mathematical formula, a set of if-else statements, etc.), a lookup table, firmware, a combination thereof, or otherwise.

FIG. 12 is a graph of central tendency versus variability (e.g., low range variability) that can be used to evaluate or identify a region or zone that holds or corresponds to a determined central tendency and variability data pair for a particular time of day (TOD). Any number of two or more zones can be used. In this embodiment the data pair can correspond to a target zone 525 or one of three hypo risk zones: a low zone 526, a moderate zone 528, or a high zone 530. A first hypo risk function (e.g., a curved or linear boundary), referred to as moderate risk function 522, differentiates between low zone 526 and moderate zone 528. A second hypo risk function, referred to as high risk function 524, differentiates between moderate zone 528 and high zone 530. The central tendency and variability data pair can be evaluated against or compared to the zones to determine a hypo risk metric for the corresponding TOD period.

The hypo risk functions 522 and 524 can be implemented in the DGA explicitly as a mathematical function (e.g., a polynomial) or can be implemented implicitly, such as by defining each zone by the pairs it contains, use of a lookup table, set of if-else statements, threshold comparisons, or otherwise. The hypo risk functions 522 and 524 can be preloaded into a software application, or can be downloaded from trusted computer system 580, or can be set by another party such as the HCP. Once implemented, the hypo risk functions 522 and 524 can be treated as fixed or can be adjusted by the user or HCP. Example methodologies for determining the hypo risk function are described in U.S. Patent Publ. No. 2018/0188400 (the '400 publication), which is incorporated by reference herein for all purposes.

At step 476, if a risk of hypoglycemia exists, then in step 480, the processor outputs an alert regarding the long hyperglycemic event and an alert regarding the risk of hypoglycemia.

At step 476, if a risk of hypoglycemia does not exist, then in step 478, the processor outputs an alert regarding the long hyperglycemic event.

Providing the notification even if a hypoglycemic risk is determined may be beneficial if the user will be available to check and act on their glucose levels frequently throughout the day, or if the person is using a system that can rapidly handle hypoglycemia risk (e.g., having a glucagon feedback system, having and automated insulin delivery system, or having a dual hormone insulin-glucagon system) and wants to further increase their time in range or time in a tight target range.

In any of the methods described, in optional steps, the estimated total duration of high glucose may be presented to the user.

In any of the methods described, in optional steps, a risk of hypoglycemia during the potential long hyperglycemic event may be determined before the alert is outputted. If a risk of hypoglycemia is determined to exist, then the alert 430 regarding the potential long hyperglycemic event may be suppressed.

One such methodology for determining a risk of hypoglycemia is described with respect to FIG. 12, showing an example embodiment of a framework for determining hypo risk and other metrics. While FIG. 12 is intended to convey the framework to the reader, however, this framework can be implemented electronically in numerous different ways, such as with a software algorithm (e.g., a mathematical formula, a set of if-else statements, etc.), a lookup table, firmware, a combination thereof, or otherwise.

FIG. 12 is a graph of central tendency versus variability (e.g., low range variability) that can be used to evaluate or identify a region or zone that holds or corresponds to a determined central tendency and variability data pair for a particular time of day (TOD). Any number of two or more zones can be used. In this embodiment the data pair can correspond to a target zone 525 or one of three hypo risk zones: a low zone 526, a moderate zone 528, or a high zone 530. A first hypo risk function (e.g., a curved or linear boundary), referred to as moderate risk function 522, differentiates between low zone 526 and moderate zone 528. A second hypo risk function, referred to as high risk function 524, differentiates between moderate zone 528 and high zone 530. The central tendency and variability data pair can be evaluated against or compared to the zones to determine a hypo risk metric for the corresponding TOD period.

The hypo risk functions 522 and 524 can be implemented in the DGA explicitly as a mathematical function (e.g., a polynomial) or can be implemented implicitly, such as by defining each zone by the pairs it contains, use of a lookup table, set of if-else statements, threshold comparisons, or otherwise. The hypo risk functions 522 and 524 can be preloaded into a software application, or can be downloaded from trusted computer system 580, or can be set by another party such as the HCP. Once implemented, the hypo risk functions 522 and 524 can be treated as fixed or can be adjusted by the user or HCP. Example methodologies for determining the hypo risk function are described in U.S. Patent Publ. No. 2018/0188400 (the '400 publication), which is incorporated by reference herein for all purposes.

In any of the methods described, in optional steps, a high glucose pattern of the potential long hyperglycemic event may be determined.

In some embodiments, determining the high glucose pattern may include determining the existence of a risk of hypoglycemia.

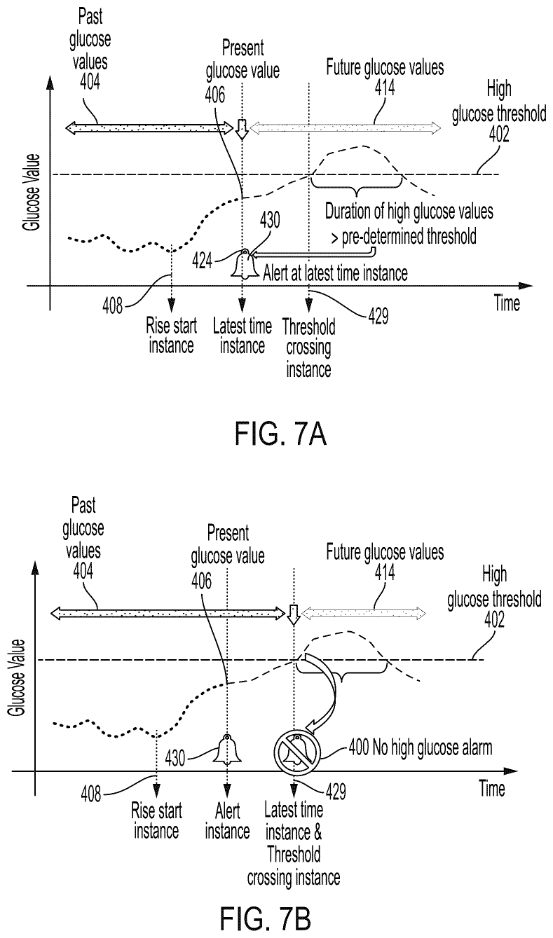

In some embodiments, at any time after the latest start of a glucose rise, the estimated total duration of high glucose may be used to categorize the high glucose pattern as a long high glucose pattern, which is a glucose pattern having high glucose levels for a long time. High glucose levels may be glucose levels of at least 140 mg/L, alternatively at least 160 mg/L, alternatively at least 180 mg/L, alternatively at least 200 mg/L, alternatively at least 220 mg/dL, alternatively at least 240 mg/dL, alternatively at least 260 mg/dL. A long time may be at least 1 hour, alternatively at least 1.5 hours, alternatively at least 2 hours, alternatively at least 2.5 hours, alternatively at least 3 hours, alternatively at least 3.5 hours, alternatively at least 4 hours. When the total duration exceeds a pre-determined threshold, the potential long hyperglycemic event may be categorized to have a long high glucose pattern, and the alert may be enunciated at the latest time instance. FIG. 7A illustrates a long high glucose pattern category having been predicted, resulting in an alert 430 at the latest time instance 424. In some embodiments, when the glucose value eventually reaches the high glucose threshold 402 at the threshold crossing instance 429, it may not be necessary to enunciate the high glucose threshold alarm 400. FIG. 7B illustrates that, after a first alert (or pre-alert) 430, a threshold crossing for the same potential long hyperglycemic event may not trigger a high glucose alarm 400.

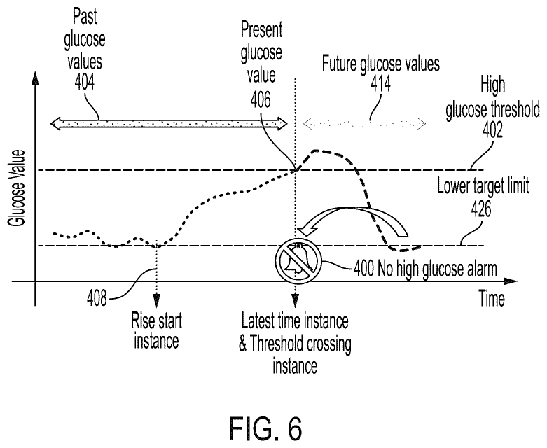

In some embodiments, the high glucose pattern may be categorized as either a long high glucose pattern or any other pattern (i.e., pattern is not a long high glucose pattern), which may include some values crossing the high glucose threshold 402 or no values crossing the high glucose threshold. Alternatively, the high glucose pattern may be grouped into a plurality of categories, such as a long high glucose pattern, a glucose pattern with low glucose risk, and any other pattern. The glucose pattern with low glucose risk may correspond to situations where any additional intervention in response to a high glucose alert may increase or cause hypoglycemia, as shown in FIG. 6, i.e., may cause their glucose levels to drop below a low glucose threshold. The low glucose threshold may be about 80 mg/dL, alternatively about 75 mg/dL, alternatively about 70 mg/dL, alternatively about 65 mg/dL. When the present glucose value is close to or crosses the high glucose threshold 402 at the threshold crossing instance 429, but the high glucose pattern is determined to have a hypoglycemic risk, the high glucose alarm 400 may be suppressed.

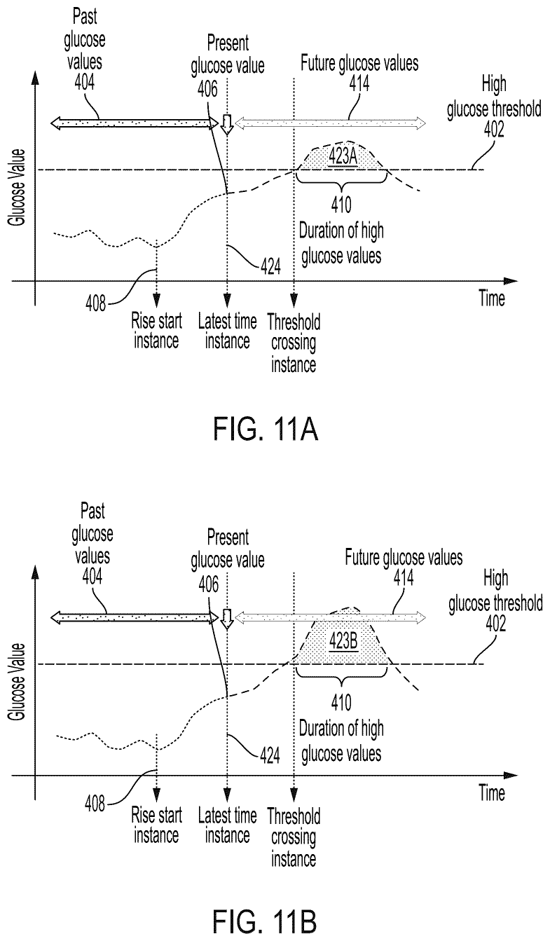

In any of the methods described, in optional steps, in addition to calculating the total duration of the potential long hyperglycemic event, AUC during the total duration may be estimated to categorize the degree of the high glucose pattern. The high glucose pattern may be categorized into low, medium, and high categories based on a pre-determined AUC threshold. As seen in FIGS. 11A-11B, the AUC for the same time period (e.g., 2 hours) can result in different AUCs depending on the glucose levels and the height of the curve. The high AUC depicted in FIG. 11B may result in higher health risks, such as a higher risk of retinopathy, than the moderate AUC depicted in FIG. 11A.

In any of the methods described, in optional steps, a time to output the alert may be determined. Because there can be more than one time instance where long high glucose is predicted, and because an earlier time instance affords more lead time to act on but less certainty, an optimal time to enunciate the alert or pre-alert 430 may be determined.

In some embodiments, a-priori association may be made between features extracted from past and present glucose values 404, 406 and an optimal alert time since the latest start of the glucose rise 408 may be determined based on a training dataset. Thus, a minimum countdown time before the alert may be enunciated, relative to the latest start of the glucose rise 408, may be determined. During the period of time from the latest start of the glucose rise 408 and including this minimum allowable time interval, an alert may not be enunciated. Afterwards, the minimum allowable time interval has passed, a first time instance categorized to predict a long high glucose pattern results in the alert to be enunciated. In some instances, this minimum allowable time may be defined based on a glucose value. For example, the first time after the latest start of the glucose rise that the glucose levels pass 140 mg/dL may be determined to be the optimal time for outputting the alert. In another embodiment, in addition to a countdown time, an expiration time may also be determined. Any time instance later than this expiration time may not result in the alert being enunciated, if an alert has not been enunciated since the countdown time. In another embodiment, as an alternative to determining an optimal alert time, the alert time may be defined as the latest time instance where the glucose value is at or near the high glucose threshold value. In this case, the estimation of the rise start instance may not be necessary.

In some embodiments, while the time instance occurs in the period between the completion of the countdown time and the start of the expiration time, the probability of long high glucose pattern is monitored, and the alert or pre-alert 430 may be enunciated as soon as the probability meets or exceeds a pre-determined threshold.

In some embodiments, the alert or pre-alert 430 is enunciated as soon as an estimated AUC exceeds a predetermined threshold. In some embodiments, the determination of the probability of the glucose pattern comprising a long high glucose pattern is determined based on calculating an area under the curve (AUC) above the high glucose threshold. For example, if the high glucose threshold is set at 180 mg/dL, then the AUC generated from glucose levels above 180 mg/dL may be compared against a pre-determined AUC threshold, or a function that generates the probability using the generated AUC value as the input. An AUC threshold example may be 80 mg/dL-hour, which is equivalent to staying 40 mg/dL above the threshold for 2 hours, or staying 20 mg/dL above the threshold for 4 hours, or other more complicated patterns. In another embodiment, instead of using the same threshold, a second higher threshold, such as 250 mg/dL, may be used to calculate the AUC even when the high glucose threshold is set at a different value, e.g., 180 mg/dL. In this example, while having at least one value crossing the 180 mg/dL is needed to determine the probability of long high glucose pattern, the probability may be low if the AUC above 250 mg/dL is zero or below a certain pre-determined AUC threshold. In another embodiment, while having at least one value crossing the 180 mg/dL is needed to determine the probability of long high glucose pattern, the probability may be low if the time spent above a second threshold, e.g., 240 mg/dL, is below a certain pre-determined time threshold.

In some embodiments, if the user has not acknowledged the alert 430, then an additional alert notification may be enunciated after a wait time. The wait time may be based on the estimated total duration such that the wait-time is short for a longer high glucose duration and longer for a shorter high glucose duration. In some embodiments, the wait time may be based on AUC-determined degree of high glucose pattern. For example, a “high” high-glucose pattern may have a shorter wait time than a moderate/low high-glucose pattern. In another embodiment, the wait time may be based on the probability of high-glucose pattern. In another embodiment, the duration and degree of high glucose pattern may be calculated again after the wait time such that if the high glucose pattern predicts a low glucose risk, then the alert 430 is not enunciated again. In some embodiments, a-priori association may be made between a long high glucose alert enunciation and low glucose risk, which may be due to user's intervention in response to the long high glucose alert. In some embodiments, a high glucose alarm 400 may be suppressed if prior acknowledgements of alert or pre-alert 430 have an associated low glucose risk.

### Exemplary Models

Development of a long hyperglycemia prediction at the moment of threshold crossing is presented as an example. A random subset of real-world CGM system data from 2014 through 2016 was selected to generate separate development and evaluation sets. The subset included 432 sensors, 10 sensors were not long enough to have at least one full hyperglycemia excursion including 4-hour data before hyperglycemia threshold. A total of 11,568 high glucose events were extracted from 422 sensors from 12 users (see Table 1 below).

| Statistics of | | | | |
|---|---|---|---|---|
| Duration | Mean | 25 Percentile | 50 Percentile | 75 Percentile |
| hour | 2.18 | 0.75 | 1.75 | 3.25 |

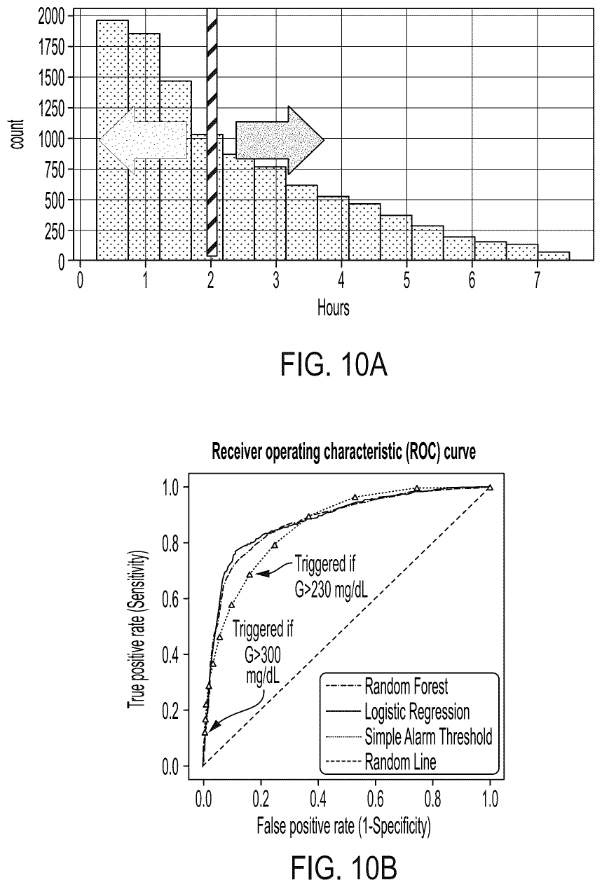

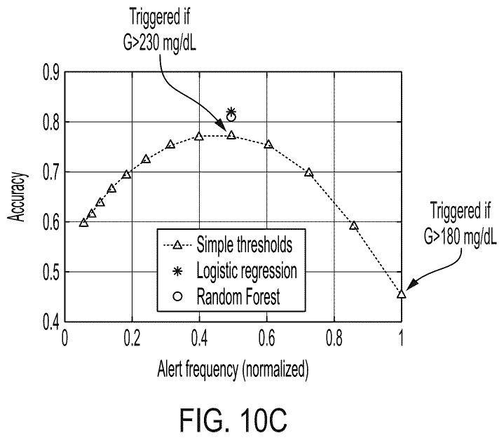

Based on the mean and median of high glucose duration, and Endocrine Society guidelines (Y. C. Kudva, et al., “Approach to Using Trend Arrows in the FreeStyle Libre Flash Glucose Monitoring Systems in Adults,” Journal of the Endocrine Society, vol. 2, pp. 1320-1337, 2018), which is expressly incorporated by reference in its entirety for all purposes, persistent high glucose may be defined to last more than 2 hours. See FIG. 10A. High glucose lasting more than 2 hours could benefit from additional insulin dose correction, while high glucose lasting less than 2 hours could present a risk of hypoglycemia if additional insulin is administered.

Some hyperglycemia excursions may have shorter or longer duration due to other factors occurring after the decision/prediction time. Examples of factors that influence the duration are additional snacks, additional insulin dose, or exercise that occur after prediction time. The efficacy of causal predictive features (model inputs) at prediction time is reduced in such cases. Principal Component Analysis and K-means clustering techniques were applied on 11,568 high glucose segments resulting in 6 distinct patterns. The outlier from each pattern were removed as they might be affected by the mentioned factors. The remaining 10695 high glucose events were grouped to 7486 segments for the development set and 32085 for the evaluation set.

Input features included recent glucose values, from 4 hours prior to threshold crossing (at 180 mg/dL), rate of change and acceleration at recent glucose values, statistics from recent glucose values, statistics from prior day, and statistics from similar time of day of prior days. Statistics from recent glucose values included Low blood glucose risk index (LBGI) & high blood glucose risk index (HBGI), net risk based on LBGI and HBGI, and mean of log transformed glucose.

Two methods and a baseline were developed: baseline (simple threshold), logistic regression, and random forest. As seen in FIG. 10B, the ROC curve shows the sensitivity-specificity trade-off for different persistent hyperglycemia detectors. The curves for the baseline and two ML methods were generated using the evaluation dataset. For the baseline detector, a high alarm set around 230 mg/dL gave the best balance of true and false alarms for detecting persistent high (>180 mg/dL). Both ML methods were superior to the baseline method based on ROC-area under the curve. In addition, ML methods made the detection call earlier than the aforementioned optimal threshold for the baseline detector (at 230 mg/dL). One pragmatic implementation was providing a more accurate detector at a similar alert frequency without incurring notification delay, as described next.

Using a CGM system with an alarm threshold at 180 mg/dL gives the accuracy=0.45 for identifying persistent hyperglycemia. The baseline detector can be optimized for accuracy by setting the threshold to 230 mg/dL, with a 50% alert frequency compared to 180 mg/dl threshold, resulting in accuracy=0.77. The cost, however, is delaying the medication intervention if needed. As seen in FIG. 10C, the proposed pre-alert, derived using either Logistic Regression or Random Forest, have accuracies of 0.82 and 0.81, respectively for the same given alert frequency, without incurring delay in notification.

The long hyperglycemic alert or pre-alert for persistently high glucose feature discussed herein can improve postmeal glycemic control by reducing the percentage of time in hyperglycemia linked to long-term complications for people with diabetes.

The long hyperglycemic alert or pre-alert may employ the user's CGM history Logistic and ML techniques to predict the persistent high glucose accurately in more than 80% of hyperglycemia events. Data used was real-world collected from 422 CGM devices.

Given the same number of alert frequency, the proposed detector has superior predictive accuracy compared to existing high glucose thresholds alarms. In addition, the predictive nature of the long hyperglycemic alert or pre-alert increases the lead time to act on hyperglycemia treatment.

### Example Embodiments of Long Hyperglycemic Alarms

Example embodiments of high glucose alarms will now be described. According to one aspect of the embodiments, a high glucose alarm may distinguish between a glucose rise event that is associated with a meal-bolus action, and one that is not associated with a recent bolus.

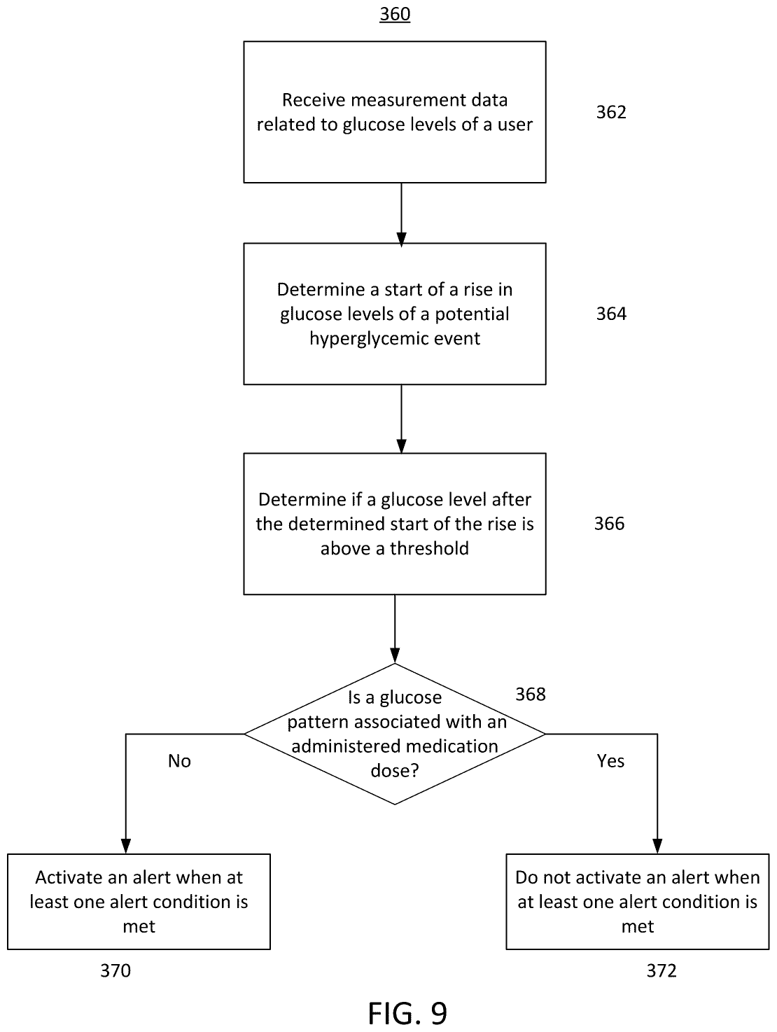

FIG. 9 depicts an example embodiment of a method 360 for outputting a high glucose alarm. At Step 362, measurement data related to glucose levels of a user are received.

At Step 364, a start of a rise in glucose levels of a potential hyperglycemic event is determined. In some embodiments, a start of a rise in glucose levels may be detected based on a calculated rate of change or glucose levels within a time period, one or more glucose levels over a threshold level, or a combination thereof.

Examples of methods for marking the start of a glucose rise are the estimation of meal start are described in US 2017/0185748, the Glucose Rate Increase Detector (GRID) method as described in Harvey, R. A. et al. “Design of the Glucose Rate Increase Detector,” J Diabetes Sci Technol. 8 (2): 307-320 (March 2014), the Variable State Dimension (VSD) method as described in Sabatini, A. M., “Variable-State-Dimension Kalman-based Filter for orientation determination using inertial and magnetic sensors,” Sensors (Basel) 12 (7): 8491-506 (2012), and methods that utilize both glucose and insulin data described in Samadi, S. et al., “Meal Detection and Carbohydrate Estimation Using Continuous Glucose Sensor Data,” IEEE J Biomed Health Inform. 21 (3): 619-627 (May 2017), all of which are hereby expressly incorporated by reference in their entireties for all purposes. In some embodiments, if a mealtime is known, it may be used as an alternative for marking the start of estimation. The delay of meal absorption, however, causes the start of a glucose rise to have a detection lag compared with actual mealtime. Although both can be marked as the start, a time shift for model inputs may be required when the same model is used for these two different cases.

At Step 366, a glucose level after the start of the rise in glucose levels is determined to be above a threshold value. The alarm may be determined based on a real-time process that may be initiated when the glucose is rising and exceeds the high glucose alert threshold, or alternatively exceeds some level below the high alert threshold (for example, 20 mg/dL below). This process may be executed periodically, but preferably whenever new glucose data has been retrieved by the system, such as every minute or every 5 minutes. The process may be performed in the user's display device (such as a mobile smart phone or a proprietary reader), or it may be performed in a web-based server associated with the smart phone. The processing may end when the user's glucose levels drop below the high alert threshold, of some other condition of glucose level and rate of change have been met such a persistent negative glucose rate of change for a period of 15 minutes. The processing may also end if the high alert has been asserted; alternatively, it may end when the high alert has been asserted and acknowledged.

At Step 368, a glucose pattern for the potential hyperglycemic event is determined to be associated with an administered medication dose or not associated with an administered medication dose.

In some embodiments, the processing may utilize a predictive model that distinguishes between glucose rise patterns associated with an insulin injection or bolus and those that are not. The output of this predictive model may be a probabilistic measure of this binary result—for example, an 80% chance that the glucose rise is associated with a recent meal-time insulin dose. This decision threshold may be configurable. Alternatively, the output may be a simple indication that the rise is or is not associated with a recent insulin dose (that is, the probabilistic thresholds are managed internal to the model). This result may drive the decision whether a high alert will be asserted. The performance of the alert logic (that is the missed detection vs. false detection percentages) may be adjusted in the predictive model by adjusting the detection thresholds used in the model. In some embodiments, missed detection of missed insulin doses may be minimized at the expense of more false alerts.

The high alert assertion logic may also include a minimum time period above the alert threshold in which the alert will be asserted regardless of the output of the predictive model. Alternatively, this design may be combined with other designs, and the decision to assert the alert may be made from a logical combination of these outputs.

Structurally, the predictive model may be an AI-based classifier. The predictive model may be a static model designed based on a population of patients, or it may be adaptive to the specific patient using the system. The system may default to the static model, but over time with use by the patient, the model may be adapted to the specific patient using the system.

A variety of techniques for developing predictive models known to those skilled in the art. In some embodiments, machine learning techniques may be used with known inputs and outputs. For the static model, inputs may include continuous glucose data and insulin delivery data for a number of patients. In some embodiments, inputs for the continuous glucose data may be obtained or derived from continuous glucose monitoring. In some embodiments, inputs for the insulin delivery data may include manual recording or automated recording by an insulin pump or connected pen. The manual inputs may be at least included in a development phase of the model and may be required for assessment of the classifier. The output may be whether or not a rise in glucose data is associated with an insulin injection or bolus, or not. The rules for this association may be, for instance, an insulin injection that occurred within about 30 minutes prior to the indication of meal start, up to the current time.

The resulting predictive model may have an input of continuous glucose and may perform pattern analysis to determine the probability of an associated insulin injection. Alternatively, the output may be glucose rises not associated with insulin injection and where the post-prandial glucose was such that a correction bolus was needed—i.e., the glucose came down reasonably quickly after the peak. This may exclude cases where food ingested by the patient was such that an insulin dose may not have been needed. Alternatively, the model may be developed using self-reported or manually logged insulin dosing information. Alternatively, the model may be developed using only continuous glucose data and not insulin delivery information. Use of a model with no medication delivery information, however, may increase the likelihood of insulin stacking.

The predictive model used may be improved over time by adapting it to a specific patient with information acquired during the patient's use of the system. The processing may be performed periodically, for instance once per week, and may be performed retrospectively as well as in real time. Adaptive modeling techniques are known to those skilled in the art. The input and outputs needed to train this model may be the same as those needed for the static model. In order to gather the insulin delivery input data, the system may retrieve the data from an insulin pump or a connected pen. Alternatively, the system may be configured for the patient to log insulin doses, or the system may estimate the insulin delivery based on patient interaction or viewing of dose guidance (such as a bolus calculator application) that may be integrated with the app or alternatively interfaced.

At Step 370, if the glucose pattern is determined not to be associated with an administered medication dose, then an alert may be activated when at least one alert condition is met. The alert condition may be met if the plurality of glucose levels after the determined start of the rise in glucose levels are above the threshold for a duration greater than a threshold. The threshold may be at least 1 hour, preferably at least 1.5 hours, more preferably at least 2 hours. The alert condition may therefore be met if the hyperglycemic event has a duration of greater than at least 1 hour, preferably at least 1.5 hours, more preferably at least 2 hours such that the hyperglycemic event is a long hyperglycemic event.

At Step 372, if the glucose pattern is determined to be associated with an administered medication dose, then an alert may not be activated when at least one alert condition is met.

### Example Embodiments of Alarm System Settings

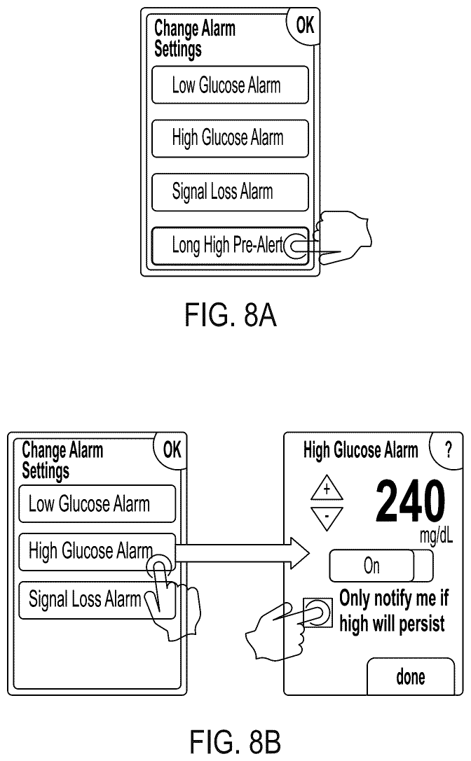

In some embodiments, an option for alerting or pre-alerting the user of long high glucose pattern is provided, such that when sufficient certainty of a sufficiently long high glucose excursion is predicted, the system can assert a pre-alert to provide the user as much lead time possible for the appropriate corrective course of action. FIG. 8A shows a long high pre-alert as an option in alarm settings.

In another embodiment, the alert or pre-alert function for long high glycemic events may be provided as an alternative to the common high glucose threshold alarm (see FIG. 8B). Choosing the pre-alert as an alternative to high glucose threshold alarm may reduce the frequency of irrelevant alarms as well as reduce the chance of rebound hypoglycemia.

In another embodiment, the high glucose alarm may be suppressed if the glucose pattern predicts low glucose risk as illustrated in FIG. 6.

Improved alarms for analyte monitoring systems are provided. For example, disclosed herein are various embodiments of methods, systems, and interfaces for long hyperglycemic alerts or pre-alerts. In addition, various embodiments of methods, systems, and interfaces for modified high glucose alerts, which predict if the high glucose episode is associated with a recently administered medication dose are described. Also, systems that include both high glucose alerts and long hyperglycemic alert options are described, in addition to enabling and disabling or suppressing certain alerts under various conditions.

Furthermore, those of skill in the art will appreciate that the embodiments described herein are not limited to the monitoring one analyte at a time, although each embodiment described herein is capable of doing so. For example, according to some embodiments, a single sensor control device can include within its housing, for example, an analyte sensor capable of sensing an in vivo glucose level and an in vivo lactate level in a bodily fluid of the user. Likewise, any and all of the aforementioned embodiments of processes, display windows, methods, and/or alarms can be configured for purposes of monitoring multiple analytes at once (e.g., glucose and lactate, glucose and ketone, etc.).

In summary, improved digital interfaces, graphical user interfaces, and alarms for analyte monitoring systems are provided. For example, disclosed herein are various embodiments of methods, systems, and interfaces for signal loss condition determination, Time-in-Ranges interfaces, GMI metrics, urgent low glucose alarms, alarm suppression features, alarm setup interfaces, and alarm unavailability detection features. In addition, various embodiments of interfaces for alarm logging and compatibility checking of an analyte monitoring software application are described. Also, various embodiments of interface enhancements are described, including an enhanced visibility mode, a voice accessibility mode, additional interfaces relating to user privacy, as well as caregiver alarms, among other embodiments.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, an analyte monitoring system includes: a sensor control device comprising an analyte sensor, wherein at least a portion of the analyte sensor is configured to be in fluid contact with a bodily fluid of a subject; and a reader device, comprising: wireless communication circuitry configured to receive data indicative of one or more glucose levels; and one or more processors coupled to a memory, the memory storing instructions that, when executed by the one or more processors, cause the one or more processors to: detect a potential long hyperglycemic event; determine a total duration for the potential long hyperglycemic event; and output an alert regarding the potential long hyperglycemic event.

In some embodiments, the potential long hyperglycemic is detected when a glucose level is above a high glucose threshold.

In some embodiments, the potential long hyperglycemic is detected when a start of a rise in glucose levels is detected.

In some embodiments, the instructions, when executed by the one or more processors, cause the one or more processors to estimate a plurality of future glucose levels.

In some embodiments, the total duration for the potential long hyperglycemic event is determined based on an amount of time future glucose levels of the plurality of future glucose levels are above a high glucose threshold. In some embodiments, the estimated future plurality of glucose levels comprises a plurality of estimated lower glucose level bounds and a plurality of upper glucose level bounds. In some embodiments, the total duration for the potential long hyperglycemic event comprises a lower bound total duration and an upper bound total duration.

In some embodiments, the total duration comprises a lower bound total duration and an upper bound total duration. In some embodiments, the lower bound total duration and the upper bound total duration are determined without determining a plurality of estimated lower glucose level bounds and a plurality of upper glucose level bounds.

In some embodiments, the total duration is estimated based on a time series of a plurality of glucose levels after the determined start of the rise.

In some embodiments, the total duration is estimated based on a plurality of glucose metrics determined from a period of time. In some embodiments, the plurality of glucose metrics comprises at least one of an average glucose value, a minimum glucose value, a maximum glucose value, and a standard deviation. In some embodiments, the period of time is selected from the group consisting of about 12 hours, about 24 hours, about 1 week, and about 2 weeks.

In some embodiments, the total duration is further estimated based on inputs selected from the group consisting of insulin doses and meals.

In some embodiments, the alert is outputted if the total duration is above a threshold value.

In some embodiments, the instructions, when executed by the one or more processors, cause the one or more processors to: determine a risk of hypoglycemia during the potential long hyperglycemic event; and if a risk of hypoglycemia is determined to exist, then suppress the alert regarding the potential long hyperglycemic event. In some embodiments, determining the risk of hypoglycemia during the potential long hyperglycemic event comprises determining a glucose pattern.

In some embodiments, the glucose pattern comprises a plurality of categories. In some embodiments, the plurality of categories comprises a long high glucose pattern, a glucose pattern with a low glucose risk, and any other pattern, and wherein a risk of hypoglyemia is determined if the glucose pattern is determined to be the glucose pattern with a low glucose risk.

In some embodiments, the instructions, when executed by the one or more processors, cause the one or more processors to: determine a probability of the glucose pattern; and determine a time to output the alert based on a comparison of the probability of the glucose pattern to a threshold probability.

In some embodiments, the instructions, when executed by the one or more processors, cause the one or more processors to determine a time to output the alert based at least on the determined total duration. In some embodiments, the potential long hyperglycemic is detected when a start of a rise in glucose levels is detected, and wherein the determined time to output the alert is after a time period after the start of the rise in glucose levels of the potential long hyperglycemic event. In some embodiments, the time period is based on a population dataset. In some embodiments, the time period is based on a dataset of the subject. In some embodiments, the time period is based on a value of a glucose level after the start of the rise in glucose levels is above a threshold value.

In some embodiments, the determined time to output the alert is when a glucose level is equal to or is above the high glucose threshold.

In some embodiments, the instructions, when executed by the one or more processors, cause the one or more processors to determine an expiration time for the alert, wherein the alert will not be outputted after the expiration time.

In some embodiments, the instructions, when executed by the one or more processors, cause the one or more processors to activate an alarm when a determined glucose level of the plurality of glucose levels is above a high glucose alarm threshold.

In some embodiments, the instructions, when executed by the one or more processors, cause the one or more processors to: determine it the subject has acknowledged the outputted alert; and output an additional alert notification after a waiting time if the subject has not acknowledged the outputted pre-alert. In some embodiments, the waiting time is based on the determined total duration.

In many embodiments, an analyte monitoring system includes: a sensor control device comprising an analyte sensor, wherein at least a portion of the analyte sensor is configured to be in fluid contact with a bodily fluid of a subject; and a reader device, comprising: wireless communication circuitry configured to receive data indicative of one ore more glucose levels; and one or more processors coupled to a memory, the memory storing instructions that, when executed by the one or more processors, cause the one or more processors to: detect a potential long hyperglycemic event; determine a plurality of future glucose levels; determine a total duration for the potential long hyperglycemic event; and output an alert regarding the potential long hyperglycemic event.

In some embodiments, the potential long hyperglycemic is detected when a glucose level is above a high glucose threshold.

In some embodiments, the potential long hyperglycemic is detected when a start of a rise in glucose levels is detected.

In some embodiments, the plurality of future glucose levels are estimated based on determined glucose levels from a predetermined time period before the start of the rise in glucose levels of the potential long hyperglycemic event.

In some embodiments, the plurality of future glucose levels are estimated based on determined glucose levels from a predetermined time period before a latest determined glucose level based on the received sensor readings. In some embodiments, the predetermined time period is about 4 hours.

In some embodiments, the total duration for the potential long hyperglycemic event is determined based on an amount of time the plurality of future glucose levels are above a high glucose threshold.

In some embodiments, the plurality of future glucose levels comprises a plurality of estimated lower glucose level bounds and a plurality of upper glucose level bounds. In some embodiments, the total duration comprises a lower bound total duration and an upper bound total duration.

In some embodiments, determining the risk of hypoglycemia during the potential long hyperglycemic event comprises determining a glucose pattern, wherein the instructions, when executed by the one or more processors, cause the one or more processors to determine an area under the curve (AUC) of the plurality of future glucose levels, wherein the glucose pattern is determined to be one of a plurality of categories based on a comparison of the AUC to a threshold AUC. In some embodiments, the plurality of categories comprises a low category, a medium category, and a high category. In some embodiments, the high glucose pattern is categorized based on an area under the curve of the estimated plurality of future glucose levels.

In some embodiments, the instructions, when executed by the one or more processors, cause the one or more processors to: determine it the subject has acknowledged the outputted alert; and output an additional alert notification after a waiting time if the subject has not acknowledged the outputted alert. In some embodiments, the waiting time is based on the determined total duration for the potential long hyperglycemic event above the high threshold.

In some embodiments, the potential long hyperglycemic is detected when a start of a rise in glucose levels is detected, and wherein the instructions, when executed by the one or more processors, cause the one or more processors to: determine an area under the curve of the plurality of future glucose levels after the start of the rise, and wherein the waiting time is based on the determined area under the curve. In some embodiments, the waiting time may be varied based on an amount of the determined area under the curve.

In some embodiments, the instructions, when executed by the one or more processors, cause the one or more processors to determine a time to output the alert based at least on the determined total duration. In some embodiments, the potential long hyperglycemic is detected when a start of a rise in glucose levels is detected, and wherein the determined time to output the alert is after a time period after the start of the rise in glucose levels of the potential long hyperglycemic event. In some embodiments, the time period is based on a population dataset. In some embodiments, the time period is based on a dataset of the subject. In some embodiments, the time period is based on a value of a glucose level after the start of the rise in glucose levels is above a threshold value.

In some embodiments, the determined time to output the alert is when a glucose level is equal to or is above the high glucose threshold.

In many embodiments, a method monitoring a glucose concentration of a user includes the steps of: receiving measurement data related to the glucose concentration of the user; determining a value of the glucose concentration of the user based on the received measurement data; in response to enablement of a high glucose alarm, activating an alarm when a determined value of the glucose concentration of the user is above a high glucose alarm threshold; in response to enablement of a long, high glucose alert, activating an alert when estimations based on the received measurement data satisfies at least one condition.

In some embodiments, the high glucose alarm is suppressed or deactivated if the long, high glucose alert is enabled. In some embodiments, the long, high glucose alert is suppressed or deactivated if the high glucose alarm is enabled. The at least one condition may be an alert condition as described herein.

In some embodiments, a method monitoring a glucose concentration of a user includes the steps of: receiving measurement data related to glucose levels of the user; detecting a potential hyperglycemic event; determining if a glucose level after the determined start of the rise is above a threshold; classifying, by one or more processors, a glucose pattern of the potential hyperglycemic event as associated with an administered medication dose or not associated with an administered medication dose; and in response to classifying the glucose pattern of the potential hyperglycemic event as not associated with the administered medication dose, activating an alert when at least one alert condition is met.

In some embodiments, the potential long hyperglycemic is detected when a glucose level is above a high glucose threshold.

In some embodiments, the potential long hyperglycemic is detected when a start of a rise in glucose levels is detected.

In some embodiments, the at least one alert condition comprises determining if a plurality of glucose levels after the start of the rise are above the threshold for a period of time.

In some embodiments, the alert is not activated in response to classifying the glucose pattern of the potential hyperglycemic event as associated with the administered medication dose.

In some embodiments, the method further includes the steps of: determining if at least one additional glucose level is above the threshold; classifying, by one or more processors, an additional glucose pattern of the potential hyperglycemic event as associated with an administered medication dose or not associated with an administered medication dose, wherein the potential hyperglycemic event comprises the start of the rise in glucose levels and the at least one additional calculated glucose level; and in response to classifying the additional glucose pattern of the potential hyperglycemic event as not associated with the administered medical dose, activating an additional alert when the at least one alert condition is met.

In some embodiments, the glucose pattern of the potential hyperglycemic event is classified using a predictive model.

In some embodiments, inputs of the predictive model comprise time-correlated glucose data.

In some embodiments, the time-correlated glucose data is received from a continuous glucose monitor.

In some embodiments, inputs of the predictive model comprise medication delivery data. In some embodiments, the medication delivery data comprises manually logged insulin dosing data. In some embodiments, the medication delivery data comprises logged insulin dosing data from a connected medication delivery device.

In some embodiments, inputs of the predictive model comprise data from a population of subjects. In some embodiments, the inputs of the predictive model further comprises data from the user.

In some embodiments, inputs of the predictive model comprise data from the user.

In some embodiments, outputs of the predictive model comprise a probability that the potential hypoglycemic event was associated with an administered medication dose.

In some embodiments, the medication dose is an insulin dose.

In some embodiments, the potential long hyperglycemic is detected when a start of a rise in glucose levels is detected, and wherein the administered medical dose was administered within about 30 minutes prior to the start of the rise in glucose levels.

In many embodiments, an analyte monitoring system includes: a sensor control device comprising an analyte sensor, wherein at least a portion of the analyte sensor is configured to be in fluid contact with a bodily fluid of a subject; and a reader device, comprising: wireless communication circuitry configured to receive data indicative of one or more glucose levels; and one or more processors coupled to a memory, the memory storing instructions that, when executed by the one or more processors, cause the one or more processors to: predict a hyperglycemic event based on the data indicative of one or more glucose levels; determine a risk of hypoglycemia after the predicted hyperglycemic event; and if a risk of hypoglycemia is determined to exist, then prevent outputting an alert regarding the predicted hyperglycemic event.

In some embodiments, the instructions further cause the one or more processors to predict a total duration of the hyperglycemic event and compare the total duration to a threshold to determine if the predicted hyperglycemic event is a long hyperglycemic event.

In some embodiments, determining the risk of hypoglycemia during the long hyperglycemic event comprises determining a glucose pattern. In some embodiments, the glucose pattern comprises a plurality of categories. In some embodiments, the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.

In some embodiments, the instructions, when executed by the one or more processors, cause the one or more processors to: determine a probability of the glucose pattern comprising a long high glucose pattern; and determine a time to output the alert based on a comparison of the probability of the glucose pattern comprising the long high glucose pattern to a threshold probability.

In some embodiments, the instructions further cause the one or more processors to: if a risk of hypoglycemia is determined not to exist, then output an alert regarding the predicted hyperglycemic event.

In many embodiments, an analyte monitoring system includes: a sensor control device comprising an analyte sensor, wherein at least a portion of the analyte sensor is configured to be in fluid contact with a bodily fluid of a subject; and a reader device, comprising: wireless communication circuitry configured to receive data indicative of one or more glucose levels; and one or more processors coupled to a memory, the memory storing instructions that, when executed by the one or more processors, cause the one or more processors to: predict a hyperglycemic event based on the data indicative of one or more glucose levels; determine a risk of hypoglycemia after the predicted hyperglycemic event; and if a risk of hypoglycemia is determined to exist, then output an alert regarding the predicted hyperglycemic event and the determined risk of hypoglycemia.

In some embodiments, the instructions further cause the one or more processors to predict a total duration of the hyperglycemic event and compare the total duration to a threshold to determine if the predicted hyperglycemic event is a long hyperglycemic event.

In some embodiments, determining the risk of hypoglycemia during the long hyperglycemic event comprises determining a glucose pattern. In some embodiments, the glucose pattern comprises a plurality of categories. In some embodiments, the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.

In some embodiments, the instructions, when executed by the one or more processors, cause the one or more processors to: determine a probability of the glucose pattern comprising a long high glucose pattern; and determine a time to output the alert based on a comparison of the probability of the glucose pattern comprising the long high glucose pattern to a threshold probability.

In some embodiments, the instructions further cause the one or more processors to: if a risk of hypoglycemia is determined not to exist, then output an alert regarding the predicted hyperglycemic event.

### Conclusion

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

### Clauses

Exemplary embodiments are set out in the following numbered clauses.
Clause 1. An analyte monitoring system, comprising:
a sensor control device comprising an analyte sensor, wherein at least a portion of the analyte sensor is configured to be in fluid contact with a bodily fluid of a subject; and
a reader device, comprising:
   wireless communication circuitry configured to receive data indicative of one or more glucose levels; and
   one or more processors coupled to a memory, the memory storing instructions that, when executed by the one or more processors, cause the one or more processors to:
      predict a hyperglycemic event based on the data indicative of one or more glucose levels;
      predict a total duration for the predicted hyperglycemic event and compare the total duration to a threshold to determine if the predicted hyperglycemic event is a long hyperglycemic event; and
      if the predicted hyperglycemic event is a long hyperglycemic event, then output an alert regarding the long hyperglycemic event.
Clause 2. The system of clause 1, wherein the hyperglycemic is predicted when a glucose level is above the threshold.
Clause 3. The system of any of clauses 1-2, wherein the hyperglycemic event is predicted when a start of a rise in glucose levels is detected.
Clause 4. The system of any of clauses 1-3, wherein the instructions, when executed by the one or more processors, cause the one or more processors to:
   estimate a plurality of future glucose levels.
Clause 5. The system of any of clauses 1-4, wherein the total duration for the predicted hyperglycemic event is determined based on an amount of time future glucose levels of the plurality of future glucose levels are above the threshold.
Clause 6. The system of any of clauses 1-5, wherein the estimated plurality of future glucose levels comprises a plurality of estimated lower glucose level bounds and a plurality of upper glucose level bounds.
Clause 7. The system of any of clauses 1-6, wherein the total duration for the predicted hyperglycemic event comprises a lower bound total duration and an upper bound total duration.
Clause 8. The system of any of clauses 1-7, wherein the total duration comprises an estimated lower bound total duration and an estimated upper bound total duration.
Clause 9. The system of any of clauses 1-8, wherein the lower bound total duration and the upper bound total duration are determined without determining a plurality of estimated lower glucose level bounds and a plurality of upper glucose level bounds.
Clause 10. The system of any of clauses 1-9, wherein the total duration for the predicted hyperglycemic event is estimated based on a time series of a plurality of glucose levels after the determined start of the rise.
Clause 11. The system of any of clauses 1-10, wherein the total duration for the predicted hyperglycemic event is estimated based on a plurality of glucose metrics determined from a period of time.
Clause 12. The system of any of clauses 1-11, wherein the plurality of glucose metrics comprises at least one of an average glucose value, a minimum glucose value, a maximum glucose value, and a standard deviation.
Clause 13. The system of any of clauses 1-12, wherein the period of time is selected from the group consisting of about 12 hours, about 24 hours, about 1 week, and about 2 weeks. Clause 14. The system of any of clauses 1-13, wherein the total duration is further estimated based on inputs selected from the group consisting of insulin doses and meals.
Clause 15. The system of any of clauses 1-14, the instructions, when executed by the one or more processors, cause the one or more processors to:
   determine a risk of hypoglycemia during the long hyperglycemic event; and
   if a risk of hypoglycemia is determined to exist, then suppress the alert regarding the long hyperglycemic event.
Clause 16. The system of any of clauses 1-15, wherein determining the risk of hypoglycemia during the long hyperglycemic event comprises determining a glucose pattern.
Clause 17. The system of any of clauses 1-16, wherein the glucose pattern comprises a plurality of categories.
Clause 18. The system of any of clauses 1-17, wherein the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.
Clause 19. The system of any of clauses 1-18, wherein the instructions, when executed by the one or more processors, cause the one or more processors to:
   determine a probability of the glucose pattern comprising a long high glucose pattern; and
   determine a time to output the alert based on a comparison of the probability of the glucose pattern comprising the long high glucose pattern to a threshold probability.
Clause 20. The system of any of clauses 1-19, wherein the instructions, when executed by the one or more processors, cause the one or more processors to:
   determine a time to output the alert based at least on the determined total duration.
Clause 21. The system of any of clauses 1-20, wherein the hyperglycemic event is predicted when a start of a rise in glucose levels is detected, and wherein the alert is outputted after a time period after the start of the rise in glucose levels.
Clause 22. The system of any of clauses 1-21, wherein the time period is based on a population dataset.
Clause 23. The system of any of clauses 1-22, wherein the time period is based on a dataset of the subject.
Clause 24. The system of any of clauses 1-23, wherein the time period is based on a value of a glucose level after the start of the rise in glucose levels is above a threshold value.
Clause 25. The system of any of clauses 1-24, wherein the determined time to output the alert is when a glucose level is equal to or is above the high glucose threshold.
Clause 26. The system of any of clauses 1-25, wherein the instructions, when executed by the one or more processors, cause the one or more processors to:
   determine an expiration time for the alert, wherein the alert will not be outputted after the expiration time.
Clause 27. The system of any of clauses 1-26, wherein the instructions, when executed by the one or more processors, cause the one or more processors to:
   activate an alarm when a determined glucose level of the one or more glucose levels is above a high glucose alarm threshold.
Clause 28. The system of any of clauses 1-27, wherein the instructions, when executed by the one or more processors, cause the one or more processors to:
   determine if the subject has acknowledged the outputted alert; and
   output an additional alert notification after a waiting time if the subject has not acknowledged the outputted alert.
Clause 29. The system of any of clauses 1-28, wherein the waiting time is based on the determined total duration.
Clause 30. The system of any of clauses 1-29, wherein the plurality of future glucose levels are estimated based on determined glucose levels from a predetermined time period before the start of the rise in glucose levels.
Clause 31. The system of any of clauses 1-30, wherein the plurality of future glucose levels are estimated based on determined glucose levels from a predetermined time period before a latest determined glucose level based on the received sensor readings.
Clause 32. The system of any of clauses 1-31, wherein the predetermined time period is about 4 hours.
Clause 33. The system of any of clauses 1-32, wherein determining the risk of hypoglycemia during the potential long hyperglycemic event comprises determining a glucose pattern, wherein the instructions, when executed by the one or more processors, cause the one or more processors to:
   determine an area under the curve (AUC) of the plurality of future glucose levels,
   wherein the glucose pattern is determined to be one of a plurality of categories based on a comparison of the AUC to a threshold AUC.
Clause 34. The system of any of clauses 1-33, wherein the plurality of categories comprises a low category, a medium category, and a high category.
Clause 35. The system of any of clauses 1-34, wherein the high glucose pattern is categorized based on an area under the curve of the estimated plurality of future glucose levels.
Clause 36. The system of any of clauses 1-35, wherein the instructions, when executed by the one or more processors, cause the one or more processors to:
   determine it the subject has acknowledged the outputted alert; and
   output an additional alert notification after a waiting time if the subject has not acknowledged the outputted alert.
Clause 37. The system of any of clauses 1-36, wherein the waiting time is based on the determined total duration for the potential long hyperglycemic event above the high threshold.
Clause 38. The system of any of clauses 1-37, wherein the potential long hyperglycemic is detected when a start of a rise in glucose levels is detected, and wherein the instructions, when executed by the one or more processors, cause the one or more processors to:
   determine an area under the curve of the plurality of future glucose levels after the start of the rise, and
   wherein the waiting time is based on the determined area under the curve.
Clause 39. These system of any of clauses 1-38, wherein the waiting time may be varied based on an amount of the determined area under the curve.
Clause 40. A method for monitoring a glucose concentration of a user, comprising:
   receiving measurement data related to the glucose concentration of the user;
   predicting a hyperglycemic event based on the data indicative of one or more glucose levels;
   predicting a total duration for the predicted hyperglycemic event and compare the total duration to a threshold to determine if the predicted hyperglycemic event is a long hyperglycemic event; and
   if the predicted hyperglycemic event is a long hyperglycemic event, then outputting an alert regarding the long hyperglycemic event.
Clause 41. The method of clause 40, wherein the hyperglycemic is predicted when a glucose level is above the threshold.
Clause 42. The method of any of clauses 40-41, wherein the hyperglycemic event is predicted when a start of a rise in glucose levels is detected.
Clause 43. The method of any of clauses 40-42, further comprising the step of estimating a plurality of future glucose levels.
Clause 44. The method of any of clauses 40-43, wherein the total duration for the predicted hyperglycemic event is determined based on an amount of time future glucose levels of the plurality of future glucose levels are above the threshold.
Clause 45. The method of any of clauses 40-44, wherein the estimated plurality of future glucose levels comprises a plurality of estimated lower glucose level bounds and a plurality of upper glucose level bounds.
Clause 46. The method of any of clauses 40-45, wherein the total duration for the predicted hyperglycemic event comprises a lower bound total duration and an upper bound total duration.
Clause 47. The method of any of clauses 40-46, wherein the wherein the total duration comprises a lower bound total duration and an upper bound total duration.
Clause 48. The method of any of clauses 40-47, wherein the lower bound total duration and the upper bound total duration are determined without determining a plurality of estimated lower glucose level bounds and a plurality of estimated upper glucose level bounds.
Clause 49. The method of any of clauses 40-48, wherein the total duration for the predicted hyperglycemic event is estimated based on a time series of a plurality of glucose levels after the determined start of the rise.
Clause 50. The method of any of clauses 40-49, wherein the total duration for the predicted hyperglycemic event is estimated based on a plurality of glucose metrics determined from a period of time.
Clause 51. The method of any of clauses 40-50, wherein the plurality of glucose metrics comprises at least one of an average glucose value, a minimum glucose value, a maximum glucose value, and a standard deviation.
Clause 52. The method of any of clauses 40-51, wherein the period of time is selected from the group consisting of about 12 hours, about 24 hours, about 1 week, and about 2 weeks.
Clause 53. The method of any of clauses 40-52, wherein the total duration is further estimated based on inputs selected from the group consisting of insulin doses and meals.
Clause 54. The method of any of clauses 40-53, further comprising the steps of:
   determining a risk of hypoglycemia during the long hyperglycemic event; and
   if a risk of hypoglycemia is determined to exist, then suppressing the alert regarding the long hyperglycemic event.
Clause 55. The method of any of clauses 40-54, wherein determining the risk of hypoglycemia during the long hyperglycemic event comprises determining a glucose pattern.
Clause 56. The method of any of clauses 40-55, wherein the glucose pattern comprises a plurality of categories.
Clause 57. The method of any of clauses 40-56, wherein the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.
Clause 58. The method of any of clauses 40-57, further comprising the steps of:
   determining a probability of the glucose pattern; and
   determining a time to output the alert based on a comparison of the probability of the glucose pattern to a threshold probability.
Clause 59. The method of any of clauses 40-58, further comprising the step of determining a time to output the alert based at least on the determined total duration.
Clause 60. The method of any of clauses 40-59, wherein the hyperglycemic event is predicted when a start of a rise in glucose levels is detected, and wherein the alert is outputted after a time period after the start of the rise in glucose levels of the predicted hyperglycemic event.
Clause 61. The method of any of clauses 40-60, wherein the time period is based on a population dataset.
Clause 62. The method of any of clauses 40-61, wherein the time period is based on a dataset of the subject.
Clause 63. The method of any of clauses 40-62, wherein the time period is based on a value of a glucose level after the start of the rise in glucose levels is above a threshold value.
Clause 64. The method of any of clauses 40-63, wherein the determined time to output the alert is when a glucose level is equal to or is above the high glucose threshold.
Clause 65. The method of any of clauses 40-64, further comprising the step of determining an expiration time for the alert, wherein the alert will not be outputted after the expiration time.
Clause 66. The method of any of clauses 40-65, further comprising the step of activating an alarm when a determined glucose level of the plurality of glucose levels is above a high glucose alarm threshold.
Clause 67. The method of any of clauses 40-66, further comprising the steps of:
   determining if the subject has acknowledged the outputted alert; and
   outputting an additional alert notification after a waiting time if the subject has not acknowledged the outputted alert.
Clause 68. The method of any of clauses 40-67, wherein the waiting time is based on the determined total duration.
Clause 69. The method of any of clauses 40-68, wherein the plurality of future glucose levels are estimated based on determined glucose levels from a predetermined time period before the start of the rise in glucose levels.
Clause 70. The method of any of clauses 40-69, wherein the plurality of future glucose levels are estimated based on determined glucose levels from a predetermined time period before a latest determined glucose level based on the received sensor readings.
Clause 71. The method of any of clauses 40-70, wherein the predetermined time period is about 4 hours.
Clause 72. The method of any of clauses 40-71, wherein determining the risk of hypoglycemia during the potential long hyperglycemic event comprises determining a glucose pattern, further comprising the step of:
   determining an area under the curve (AUC) of the plurality of future glucose levels,
   wherein the glucose pattern is determined to be one of a plurality of categories based on a comparison of the AUC to a threshold AUC.
Clause 73. The method of any of clauses 40-72, wherein the plurality of categories comprises a low category, a medium category, and a high category.
Clause 74. The method of any of clauses 40-73, wherein the high glucose pattern is categorized based on an area under the curve of the estimated plurality of future glucose levels.
Clause 75. The method of any of clauses 40-74, further comprising the steps of
   determining if the subject has acknowledged the outputted alert; and
   outputting an additional alert notification after a waiting time if the subject has not acknowledged the outputted alert.
Clause 76. The method of any of clauses 40-75, wherein the waiting time is based on the determined total duration for the potential long hyperglycemic event above the high threshold.
Clause 77. The method of any of clauses 40-76, wherein the potential long hyperglycemic is detected when a start of a rise in glucose levels is detected, and wherein the method further comprises the step of:
   determining an area under the curve of the plurality of future glucose levels after the start of the rise, and
   wherein the waiting time is based on the determined area under the curve.
Clause 78. The method of any of clauses 40-77, wherein the waiting time may be varied based on an amount of the determined area under the curve.
Clause 79. An analyte monitoring system, comprising:
a sensor control device comprising an analyte sensor, wherein at least a portion of the analyte sensor is configured to be in fluid contact with a bodily fluid of a subject; and
a reader device, comprising:
   wireless communication circuitry configured to receive data indicative of one ore more glucose levels; and
   one or more processors coupled to a memory, the memory storing instructions that, when executed by the one or more processors, cause the one or more processors to:
      predict a hyperglycemic event based on the data indicative of one or more glucose levels;
         estimate a plurality of future glucose levels;
      predict a total duration for the hyperglycemic event and compare the total duration to a threshold to determine if the predicted hyperglycemic event is a long hyperglycemic event; and
      if the predicted hyperglycemic event is a long hyperglycemic event, then output an alert regarding the long hyperglycemic event.
Clause 80. The system of clause 79, wherein the hyperglycemic event is predicted when a glucose level is above the threshold.
Clause 81. The system of any of clauses 79-80, wherein the hyperglycemic event is predicted when a start of a rise in glucose levels is detected.
Clause 82. The system of any of clauses 79-81, wherein the plurality of future glucose levels are estimated based on determined glucose levels from a predetermined time period before the start of the rise in glucose levels.
Clause 83. The system of any of clauses 79-82, wherein the plurality of future glucose levels are estimated based on determined glucose levels from a predetermined time period before a latest determined glucose level based on the received sensor readings.
Clause 84. The system of any of clauses 79-83, wherein the predetermined time period is about 4 hours.
Clause 85. The system of any of clauses 79-84, wherein the total duration for the hyperglycemic event is predicted based on an amount of time the plurality of future glucose levels are above the threshold.
Clause 86. The system of any of clauses 79-85, wherein the plurality of future glucose levels comprises a plurality of estimated lower glucose level bounds and a plurality of estimated upper glucose level bounds.
Clause 87. The system of any of clauses 79-86, wherein the total duration comprises a lower bound total duration and an upper bound total duration.
Clause 88. The system of any of clauses 79-87, wherein the instructions, when executed by the one or more processors, cause the one or more processors to:
   determine a risk of hypoglycemia during the long hyperglycemic event; and
   if a risk of hypoglycemia is determined to exist, then suppress the alert regarding the long hyperglycemic event.
Clause 89. The system of any of clauses 79-88, wherein determining the risk of hypoglycemia during the potential long hyperglycemic event comprises determining a glucose pattern, wherein the instructions, when executed by the one or more processors, cause the one or more processors to:
   determine an area under the curve (AUC) of the plurality of future glucose levels,
   wherein the glucose pattern is categorized according to one of a plurality of categories based on a comparison of the AUC to a threshold AUC.
Clause 90. The system of any of clauses 79-89, wherein the plurality of categories comprises a low category, a medium category, and a high category.
Clause 91. The system of any of clauses 79-90, wherein the glucose pattern is categorized as a high glucose pattern based on an area under the curve of the estimated plurality of future glucose levels.
Clause 92. The system of any of clauses 79-91, wherein the instructions, when executed by the one or more processors, cause the one or more processors to:
   determine it the subject has acknowledged the outputted alert; and
   output an additional alert notification after a waiting time if the subject has not acknowledged the outputted alert.
Clause 93. The system of any of clauses 79-92, wherein the waiting time is based on the determined total duration for the potential long hyperglycemic event above a high threshold.
Clause 94. The system of any of clauses 79-93, wherein the hyperglycemic alert is predicted when a start of a rise in glucose levels is detected, and wherein the instructions, when executed by the one or more processors, cause the one or more processors to:
   determine an area under the curve of the plurality of future glucose levels after the start of the rise, and
   wherein the waiting time is based on the determined area under the curve.
Clause 95. The system of any of clauses 79-94, wherein the waiting time may be varied based on an amount of the determined area under the curve.
Clause 96. The system of any of clauses 79-95, wherein the instructions, when executed by the one or more processors, cause the one or more processors to:
   determine a time to output the alert based at least on the determined total duration.
Clause 97. The system of any of clauses 79-96, wherein the hyperglycemic event is detected when a start of a rise in glucose levels is detected, and wherein the determined time to output the alert is after a time period after the start of the rise in glucose levels of the potential long hyperglycemic event.
Clause 98. The system of any of clauses 79-97, wherein the time period is based on a population dataset.
Clause 99. The system of any of clauses 79-98, wherein the time period is based on a dataset of the subject.
Clause 100. The system of any of clauses 79-99, wherein the time period is based on a value of a glucose level after the start of the rise in glucose levels is above a threshold value.
Clause 101. The system of any of clauses 79-100, wherein the determined time to output the alert is when a glucose level is equal to or is above the high glucose threshold.
Clause 102. A method for monitoring a glucose concentration of a user, comprising:
   receiving measurement data related to the glucose concentration of the user;
   predicting a hyperglycemic event based on the data indicative of one or more glucose levels;
   estimating a plurality of future glucose levels;
   predicting a total duration for the predicted hyperglycemic event and compare the total duration to a threshold to determine if the predicted hyperglycemic event is a long hyperglycemic event; and
   if the predicted hyperglycemic event is a long hyperglycemic event, then outputting an alert regarding the long hyperglycemic event.
Clause 103. The method of clause 102, wherein the hyperglycemic event is predicted when a glucose level is above the threshold.
Clause 104. The method of any of clauses 102-103, wherein the hyperglycemic event is predicted when a start of a rise in glucose levels is detected.
Clause 105. The method of any of clauses 102-104, wherein the plurality of future glucose levels are estimated based on determined glucose levels from a predetermined time period before the start of the rise in glucose levels of the hyperglycemic event.
Clause 106. The method of any of clauses 102-105, wherein the plurality of future glucose levels are estimated based on determined glucose levels from a predetermined time period before a latest determined glucose level based on the received sensor readings.
Clause 107. The method of any of clauses 102-106, wherein the predetermined time period is about 4 hours.
Clause 108. The method of any of clauses 102-107, wherein the total duration for the hyperglycemic event is predicted based on an amount of time the plurality of future glucose levels are above the threshold.
Clause 109. The method of any of clauses 102-108, wherein the plurality of future glucose levels comprises a plurality of estimated lower glucose level bounds and a plurality of upper glucose level bounds.
Clause 110. The method of any of clauses 102-109, wherein the total duration comprises a lower bound total duration and an upper bound total duration.
Clause 111. The method of any of clauses 102-110, further comprising the step of:
   determining a risk of hypoglycemia during the long hyperglycemic event; and
   if a risk of hypoglycemia is determined to exist, then suppressing the alert regarding the long hyperglycemic event.
Clause 112. The method of any of clauses 102-111, wherein determining the risk of hypoglycemia during the potential long hyperglycemic event comprises determining a glucose pattern, wherein the method further comprises the step of:
   determining an area under the curve (AUC) of the plurality of future glucose levels,
   wherein the glucose pattern is determined to be one of a plurality of categories based on a comparison of the AUC to a threshold AUC.
Clause 113. The method of any of clauses 102-112, wherein the plurality of categories comprises a low category, a medium category, and a high category.
Clause 114. The method of any of clauses 102-113, wherein the high glucose pattern is categorized based on an area under the curve of the estimated plurality of future glucose levels.
Clause 115. The method of any of clauses 102-114, wherein the method further includes the steps of:
   determining it the subject has acknowledged the outputted alert; and
   outputting an additional alert notification after a waiting time if the subject has not acknowledged the outputted alert.
Clause 116. The method of any of clauses 102-115, wherein the waiting time is based on the determined total duration for the potential long hyperglycemic event above the high threshold.
Clause 117. The method of any of clauses 102-116, wherein the hyperglycemic alert is predicted when a start of a rise in glucose levels is detected, and wherein the instructions, when executed by the one or more processors, cause the one or more processors to:
   determine an area under the curve of the plurality of future glucose levels after the start of the rise, and
   wherein the waiting time is based on the determined area under the curve.
Clause 118. The method of any of clauses 102-117, wherein the waiting time may be varied based on an amount of the determined area under the curve.
Clause 119. The method of any of clauses 102-118, wherein the instructions, when executed by the one or more processors, cause the one or more processors to:
   determine a time to output the alert based at least on the determined total duration.
Clause 120. The method of any of clauses 102-119, wherein the hyperglycemic event is detected when a start of a rise in glucose levels is detected, and wherein the determined time to output the alert is after a time period after the start of the rise in glucose levels of the potential long hyperglycemic event.
Clause 121. The method of any of clauses 102-120, wherein the time period is based on a population dataset.
Clause 122. The method of any of clauses 102-121, wherein the time period is based on a dataset of the subject.
Clause 123. The method of any of clauses 102-122, wherein the time period is based on a value of a glucose level after the start of the rise in glucose levels is above a threshold value.
Clause 124. The method of any of clauses 102-123, wherein the determined time to output the alert is when a glucose level is equal to or is above the high glucose threshold.
Clause 125. A method for monitoring a glucose concentration of a user, comprising:
   receiving measurement data related to the glucose concentration of the user;
   determining a value of the glucose concentration of the user based on the received measurement data;
   in response to enablement of a high glucose alarm, outputting an alarm when a determined value of the glucose concentration of the user is above a high glucose alarm threshold; and
   in response to enablement of a long hyperglycemic alert, outputting an alert when estimations based on the received measurement data satisfies at least one condition.
Clause 126. The system of clause 125, wherein the high glucose alarm is suppressed if the long hyperglycemic alert is enabled.
Clause 127. The system of any of clauses 125-126, wherein the long hyperglycemic alert is suppressed if the high glucose alarm is enabled.
Clause 128. The system of any of clauses 125-127, wherein the method further comprises the steps of:
   predicting a hyperglycemic event based on the data indicative of one or more glucose levels;
   predicting a total duration for the predicted hyperglycemic event; and
   wherein the at least one condition comprises determining if the predicted total duration for the predicted hyperglycemic event is greater than a threshold duration.
Clause 129. A method for monitoring a glucose concentration of a user, comprising:
   receiving measurement data indicative of one or more glucose levels of the user;
   predicting a hyperglycemic event based on the data indicative of one or more glucose levels, wherein the step of predicting the hyperglycemic event comprises:
      determining a start of a rise in glucose levels; and
      determining if a glucose level after a start of a rise in glucose levels is above a threshold;
classifying, by one or more processors, a glucose pattern of the predicted hyperglycemic event as associated with an administered medication dose or not associated with an administered medication dose; and
   in response to classifying the glucose pattern of the predicted hyperglycemic event as not associated with the administered medication dose, activating an alert when at least one alert condition is met.
Clause 130. The method of clause 129, wherein the hyperglycemic event is predicted when a glucose level after the start of the rise in glucose levels is above a high glucose threshold.
Clause 131. The method of any of clauses 129-130, wherein the hyperglycemic event is predicted when the start of a rise in glucose levels is detected.
Clause 132. The method of any of clauses 129-131, wherein the at least one alert condition comprises determining if a plurality of glucose levels after the start of the rise are above the threshold for a period of time.
Clause 133. The method of any of clauses 129-132, wherein the alert is not activated in response to classifying the glucose pattern of the potential hyperglycemic event as associated with the administered medication dose.
Clause 134. The method of any of clauses 129-133, further comprising the steps of:
   determining if at least one additional glucose level is above the threshold;
   classifying, by one or more processors, an additional glucose pattern of the predicted hyperglycemic event as associated with an administered medication dose or not associated with an administered medication dose, wherein the predicted hyperglycemic event comprises the start of the rise in glucose levels and the at least one additional calculated glucose level; and
   in response to classifying the additional glucose pattern of the predicted hyperglycemic event as not associated with the administered medical dose, activating an additional alert when the at least one alert condition is met.
Clause 135. The method of any of clauses 129-134, wherein the glucose pattern of the predicted hyperglycemic event is classified using a predictive model.
Clause 136. The method of any of clauses 129-135, wherein inputs of the predictive model comprise time-correlated glucose data.
Clause 137. The method of any of clauses 129-136, wherein the time-correlated glucose data is received from a continuous glucose monitor.
Clause 138. The method of any of clauses 129-137, wherein inputs of the predictive model comprise medication delivery data.
Clause 139. The method of any of clauses 129-138, wherein the medication delivery data comprises manually logged insulin dosing data.
Clause 140. The method of any of clauses 129-139, wherein the medication delivery data comprises logged insulin dosing data from a connected medication delivery device.
Clause 141. The method of any of clauses 129-140, wherein inputs of the predictive model comprise data from a population of subjects.
Clause 142. The method of any of clauses 129-141, wherein the inputs of the predictive model further comprises data from the user.
Clause 143. The method of any of clauses 129-142, wherein inputs of the predictive model comprise data from the user.
Clause 144. The method of any of clauses 129-143, wherein outputs of the predictive model comprise a probability that the potential hypoglycemic event was associated with an administered medication dose.
Clause 145. The method of any of clauses 129-144, wherein the medication dose is an insulin dose.
Clause 146. The method of any of clauses 129-145, wherein the long hyperglycemic is predicted when a start of a rise in glucose levels is detected, and wherein the administered medical dose was administered within about 30 minutes prior to the start of the rise in glucose levels.
Clause 147. An analyte monitoring system, comprising:
a sensor control device comprising an analyte sensor, wherein at least a portion of the analyte sensor is configured to be in fluid contact with a bodily fluid of a subject; and
a reader device, comprising:
   wireless communication circuitry configured to receive data indicative of one or more glucose levels; and
   one or more processors coupled to a memory, the memory storing instructions that, when executed by the one or more processors, cause the one or more processors to:
      predict a hyperglycemic event based on the data indicative of one or more glucose levels;
      determine a risk of hypoglycemia after the predicted hyperglycemic event; and
      if a risk of hypoglycemia is determined to exist, then prevent outputting an alert regarding the predicted hyperglycemic event.
Clause 148. The system of clause 147, wherein the instructions further cause the one or more processors to:
predict a total duration of the hyperglycemic event and compare the total duration to a threshold to determine if the predicted hyperglycemic event is a long hyperglycemic event.
Clause 149. The system of any of clauses 147-148, wherein determining the risk of hypoglycemia during the long hyperglycemic event comprises determining a glucose pattern.
Clause 150. The system of clause 149, wherein the glucose pattern comprises a plurality of categories.
Clause 151. The system of clause 150, wherein the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.
Clause 152. The system of any of clauses 149-151, wherein the instructions, when executed by the one or more processors, cause the one or more processors to:
   determine a probability of the glucose pattern comprising a long high glucose pattern; and
   determine a time to output the alert based on a comparison of the probability of the glucose pattern comprising the long high glucose pattern to a threshold probability.
Clause 153. The system of any of clauses 147-152, wherein the instructions further cause the one or more processors to:
   if a risk of hypoglycemia is determined not to exist, then output an alert regarding the predicted hyperglycemic event.
Clause 154. An analyte monitoring system, comprising:
a sensor control device comprising an analyte sensor, wherein at least a portion of the analyte sensor is configured to be in fluid contact with a bodily fluid of a subject; and
a reader device, comprising:
   wireless communication circuitry configured to receive data indicative of one or more glucose levels; and
   one or more processors coupled to a memory, the memory storing instructions that, when executed by the one or more processors, cause the one or more processors to:
      predict a hyperglycemic event based on the data indicative of one or more glucose levels;
      determine a risk of hypoglycemia after the predicted hyperglycemic event; and
      if a risk of hypoglycemia is determined to exist, then output an alert regarding the predicted hyperglycemic event and the determined risk of hypoglycemia.
Clause 155. The system of clause 154, wherein the instructions further cause the one or more processors to:
predict a total duration of the hyperglycemic event and compare the total duration to a threshold to determine if the predicted hyperglycemic event is a long hyperglycemic event.
Clause 156. The system of any of clauses 154-155, wherein determining the risk of hypoglycemia during the long hyperglycemic event comprises determining a glucose pattern.
Clause 157. The system of clause 156, wherein the glucose pattern comprises a plurality of categories.
Clause 158. The system of clause 157, wherein the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.
Clause 159. The system of clause 156, wherein the instructions, when executed by the one or more processors, cause the one or more processors to:
   determine a probability of the glucose pattern comprising a long high glucose pattern; and
   determine a time to output the alert based on a comparison of the probability of the glucose pattern comprising the long high glucose pattern to a threshold probability.
Clause 160. The system of any of clauses 154-159, wherein the instructions further cause the one or more processors to:
   if a risk of hypoglycemia is determined not to exist, then output an alert regarding the predicted hyperglycemic event.

1. A method for monitoring a glucose concentration of a user, comprising:
receiving measurement data related to the glucose concentration of the user;
determining a value of the glucose concentration of the user based on the received measurement data;
in response to enablement of a high glucose alarm, outputting an alarm when a determined value of the glucose concentration of the user is above a high glucose alarm threshold; and
in response to enablement of a long hyperglycemic alert, outputting an alert when estimations based on the received measurement data satisfies at least one condition.

2. The system of claim 1, wherein the high glucose alarm is suppressed if the long hyperglycemic alert is enabled.

3. The system of claim 1, wherein the long hyperglycemic alert is suppressed if the high glucose alarm is enabled.

4. The system of claim 1, wherein the method further comprises the steps of:
predicting a hyperglycemic event based on the data indicative of one or more glucose levels;
predicting a total duration for the predicted hyperglycemic event; and
wherein the at least one condition comprises determining if the predicted total duration for the predicted hyperglycemic event is greater than a threshold duration.

5. An analyte monitoring system, comprising:
a sensor control device comprising an analyte sensor, wherein at least a portion of the analyte sensor is configured to be in fluid contact with a bodily fluid of a subject; and
a reader device, comprising:
wireless communication circuitry configured to receive data indicative of one or more glucose levels; and
one or more processors coupled to a memory, the memory storing instructions that, when executed by the one or more processors, cause the one or more processors to:
predict a hyperglycemic event based on the data indicative of one or more glucose levels;
determine a risk of hypoglycemia after the predicted hyperglycemic event; and
if a risk of hypoglycemia is determined to exist, then prevent outputting an alert regarding the predicted hyperglycemic event.

6. The system of claim 5, wherein the instructions further cause the one or more processors to:
predict a total duration of the hyperglycemic event and compare the total duration to a threshold to determine if the predicted hyperglycemic event is a long hyperglycemic event.

7. The system of claim 5, wherein determining the risk of hypoglycemia during the long hyperglycemic event comprises determining a glucose pattern.

8. The system of claim 7, wherein the glucose pattern comprises a plurality of categories.

9. The system of claim 8, wherein the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.

10. The system of claim 7, wherein the instructions, when executed by the one or more processors, cause the one or more processors to:
determine a probability of the glucose pattern comprising a long high glucose pattern; and
determine a time to output the alert based on a comparison of the probability of the glucose pattern comprising the long high glucose pattern to a threshold probability.

11. The system of claim 5, wherein the instructions further cause the one or more processors to:
if a risk of hypoglycemia is determined not to exist, then output an alert regarding the predicted hyperglycemic event.

12-50. (canceled)

51. An analyte monitoring system, comprising:
a sensor control device comprising an analyte sensor, wherein at least a portion of the analyte sensor is configured to be in fluid contact with a bodily fluid of a subject; and
a reader device, comprising:
wireless communication circuitry configured to receive data indicative of one or more glucose levels; and
one or more processors coupled to a memory, the memory storing instructions that, when executed by the one or more processors, cause the one or more processors to:
predict a hyperglycemic event based on the data indicative of one or more glucose levels;
determine a risk of hypoglycemia after the predicted hyperglycemic event; and
if a risk of hypoglycemia is determined to exist, then output an alert regarding the predicted hyperglycemic event and the determined risk of hypoglycemia.

52. The system of claim 51, wherein the instructions further cause the one or more processors to:
predict a total duration of the hyperglycemic event and compare the total duration to a threshold to determine if the predicted hyperglycemic event is a long hyperglycemic event.

53. The system of claim 51, wherein determining the risk of hypoglycemia during the long hyperglycemic event comprises determining a glucose pattern.

54. The system of claim 53, wherein the glucose pattern comprises a plurality of categories.

55. The system of claim 54, wherein the plurality of categories comprises a long high glucose pattern, a glucose pattern with a low glucose risk, and any other pattern, and wherein a risk of hypoglycemia is determined if the glucose pattern is determined to be the glucose pattern with a low glucose risk.

56. The system of claim 53, wherein the instructions, when executed by the one or more processors, cause the one or more processors to:
determine a probability of the glucose pattern comprising a long high glucose pattern; and
determine a time to output the alert based on a comparison of the probability of the glucose pattern comprising the long high glucose pattern to a threshold probability.

57. The system of claim 51, wherein the instructions further cause the one or more processors to:
if a risk of hypoglycemia is determined not to exist, then output an alert regarding the predicted hyperglycemic event.

58-114. (canceled)
